SEQUENCE LISTING (14 sequences)

SEQ ID NO: 1 - DNA, 912 bp, Pantoea  stewartii
ttgacggtctgcgcaaaaaaacacgttcaccttactggcatttcggctgagcagttgctggctgatatcgatagccgccttgatcagttactgccggttcagggtgagcgggattgtgtgggtgccgcgatgcgtgaaggcacgctggcaccgggcaaacgtattcgtccgatgctgctgttattaacagcgcgcgatcttggctgtgcgatcagtcacgggggattactggatttagcctgcgcggttgaaatggtgcatgctgcctcgctgattctggatgatatgccctgcatggacgatgcgcagatgcgtcgggggcgtcccaccattcacacgcagtacggtgaacatgtggcgattctggcggcggtcgctttactcagcaaagcgtttggggtgattgccgaggctgaaggtctgacgccgatagccaaaactcgcgcggtgtcggagctgtccactgcgattggcatgcagggtctggttcagggccagtttaaggacctctcggaaggcgataaaccccgcagcgccgatgccatactgctaaccaatcagtttaaaaccagcacgctgttttgcgcgtcaacgcaaatggcgtccattgcggccaacgcgtcctgcgaagcgcgtgagaacctgcatcgtttctcgctcgatctcggccaggcctttcagttgcttgacgatcttaccgatggcatgaccgataccggcaaagacatcaatcaggatgcaggtaaatcaacgctggtcaatttattaggctcaggcgcggtcgaagaacgcctgcgacagcatttgcgcctggccagtgaacacctttccgcggcatgccaaaacggccattccaccacccaactttttattcaggcctggtttgacaaaaaactcgctgccgtcagttaa

SEQ ID NO: 2 - protein, 303 aa, Pantoea  stewartii
LTVCAKKHVHLTGISAEQLLADIDSRLDQLLPVQGERDCVGAAMREGTLAPGKRIRPMLLLLTARDLGCAISHGGLLDLACAVEMVHAASLILDDMPCMDDAQMRRGRPTIHTQYGEHVAILAAVALLSKAFGVIAEAEGLTPIAKTRAVSELSTAIGMQGLVQGQFKDLSEGDKPRSADAILLTNQFKTSTLFCASTQMASIAANASCEARENLHRFSLDLGQAFQLLDDLTDGMTDTGKDINQDAGKSTLVNLLGSGAVEERLRQHLRLASEHLSAACQNGHSTTQLFIQAWFDKKLAAVS

SEQ ID NO: 3 - DNA, 1296 bp, Pantoea  stewartii
atgagccattttgcggtgatcgcaccgccctttttcagccatgttcgcgctctgcaaaaccttgctcaggaattagtggcccgcggtcatcgtgttacgttttttcagcaacatgactgcaaagcgctggtaacgggcagcgatatcggattccagaccgtcggactgcaaacgcatcctcccggttccttatcgcacctgctgcacctggccgcgcacccactcggaccctcgatgttacgactgatcaatgaaatggcacgtaccagcgatatgctttgccgggaactgcccgccgcttttcatgcgttgcagatagagggcgtgatcgttgatcaaatggagccggcaggtgcagtagtcgcagaagcgtcaggtctgccgtttgtttcggtggcctgcgcgctgccgctcaaccgcgaaccgggtttgcctctggcggtgatgcctttcgagtacggcaccagcgatgcggctcgggaacgctataccaccagcgaaaaaatttatgactggctgatgcgacgtcacgatcgtgtgatcgcgcatcatgcatgcagaatgggtttagccccgcgtgaaaaactgcatcattgtttttctccactggcacaaatcagccagttgatccccgaactggattttccccgcaaagcgctgccagactgctttcatgcggttggaccgttacggcaaccccaggggacgccggggtcatcaacttcttattttccgtccccggacaaaccccgtatttttgcctcgctgggcaccctgcagggacatcgttatggcctgttcaggaccatcgccaaagcctgcgaagaggtggatgcgcagttactgttggcacactgtggcggcctctcagccacgcaggcaggtgaactggcccggggcggggacattcaggttgtggattttgccgatcaatccgcagcactttcacaggcacagttgacaatcacacatggtgggatgaatacggtactggacgctattgcttcccgcacaccgctactggcgctgccgctggcatttgatcaacctggcgtggcatcacgaattgtttatcatggcatcggcaagcgtgcgtctcggtttactaccagccatgcgctggcgcggcagattcgatcgctgctgactaacaccgattacccgcagcgtatgacaaaaattcaggccgcattgcgtctggcaggcggcacaccagccgccgccgatattgttgaacaggcgatgcggacctgtcagccagtactcagtgggcaggattatgcaaccgcactatga

SEQ ID NO: 4 - protein, 431 aa, Pantoea  stewartii
MSHFAVIAPPFFSHVRALQNLAQELVARGHRVTFFQQHDCKALVTGSDIGFQTVGLQTHPPGSLSHLLHLAAHPLGPSMLRLINEMARTSDMLCRELPAAFHALQIEGVIVDQMEPAGAVVAEASGLPFVSVACALPLNREPGLPLAVMPFEYGTSDAARERYTTSEKIYDWLMRRHDRVIAHHACRMGLAPREKLHHCFSPLAQISQLIPELDFPRKALPDCFHAVGPLRQPQGTPGSSTSYFPSPDKPRIFASLGTLQGHRYGLFRTIAKACEEVDAQLLLAHCGGLSATQAGELARGGDIQVVDFADQSAALSQAQLTITHGGMNTVLDAIASRTPLLALPLAFDQPGVASRIVYHGIGKRASRFTTSHALARQIRSLLTNTDYPQRMTKIQAALRLAGGTPAAADIVEQAMRTCQPVLSGQDYATAL

SEQ ID NO: 5 - DNA, 1149 bp, Pantoea  stewartii
atgcaaccgcactatgatctcattctggtcggtgccggtctggctaatggccttatcgcgctccggcttcagcaacagcatccggatatgcggatcttgcttattgaggcgggtcctgaggcgggagggaaccatacctggtcctttcacgaagaggatttaacgctgaatcagcatcgctggatagcgccgcttgtggtccatcactggcccgactaccaggttcgtttcccccaacgccgtcgccatgtgaacagtggctactactgcgtgacctcccggcatttcgccgggatactccggcaacagtttggacaacatttatggctgcataccgcggtttcagccgttcatgctgaatcggtccagttagcggatggccggattattcatgccagtacagtgatcgacggacggggttacacgcctgattctgcactacgcgtaggattccaggcatttatcggtcaggagtggcaactgagcgcgccgcatggtttatcgtcaccgattatcatggatgcgacggtcgatcagcaaaatggctaccgctttgtttataccctgccgctttccgcaaccgcactgctgatcgaagacacacactacattgacaaggctaatcttcaggccgaacgggcgcgtcagaacattcgcgattatgctgcgcgacagggttggccgttacagacgttgctgcgggaagaacagggtgcattgcccattacgttaacgggcgataatcgtcagttttggcaacagcaaccgcaagcctgtagcggattacgcgccgggctgtttcatccgacaaccggctactccctaccgctcgcggtggcgctggccgatcgtctcagcgcgctggatgtgtttacctcttcctctgttcaccagacgattgctcactttgcccagcaacgttggcagcaacaggggtttttccgcatgctgaatcgcatgttgtttttagccggaccggccgagtcacgctggcgtgtgatgcagcgtttctatggcttacccgaggatttgattgcccgcttttatgcgggaaaactcaccgtgaccgatcggctacgcattctgagcggcaagccgcccgttcccgttttcgcggcattgcaggcaattatgacgactcatcgttga

SEQ ID NO: 6 - protein, 382 aa, Pantoea  stewartii
MQPHYDLILVGAGLANGLIALRLQQQHPDMRILLIEAGPEAGGNHTWSFHEEDLTLNQHRWIAPLVVHHWPDYQVRFPQRRRHVNSGYYCVTSRHFAGILRQQFGQHLWLHTAVSAVHAESVQLADGRIIHASTVIDGRGYTPDSALRVGFQAFIGQEWQLSAPHGLSSPIIMDATVDQQNGYRFVYTLPLSATALLIEDTHYIDKANLQAERARQNIRDYAARQGWPLQTLLREEQGALPITLTGDNRQFWQQQPQACSGLRAGLFHPTTGYSLPLAVALADRLSALDVFTSSSVHQTIAHFAQQRWQQQGFFRMLNRMLFLAGPAESRWRVMQRFYGLPEDLIARFYAGKLTVTDRLRILSGKPPVPVFAALQAIMTTHR

SEQ ID NO: 7 - DNA, 1479 bp, Pantoea  stewartii
atgaaaccaactacggtaattggtgcgggctttggtggcctggcactggcaattcgtttacaggccgcaggtattcctgttttgctgcttgagcagcgcgacaagccgggtggccgggcttatgtttatcaggagcagggctttacttttgatgcaggccctaccgttatcaccgatcccagcgcgattgaagaactgtttgctctggccggtaaacagcttaaggattacgtcgagctgttgccggtcacgccgttttatcgcctgtgctgggagtccggcaaggtcttcaattacgataacgaccaggcccagttagaagcgcagatacagcagtttaatccgcgcgatgttgcgggttatcgagcgttccttgactattcgcgtgccgtattcaatgagggctatctgaagctcggcactgtgccttttttatcgttcaaagacatgcttcgggccgcgccccagttggcaaagctgcaggcatggcgcagcgtttacagtaaagttgccggctacattgaggatgagcatcttcggcaggcgttttcttttcactcgctcttagtgggggggaatccgtttgcaacctcgtccatttatacgctgattcacgcgttagaacgggaatggggcgtctggtttccacgcggtggaaccggtgcgctggtcaatggcatgatcaagctgtttcaggatctgggcggcgaagtcgtgcttaacgcccgggtcagtcatatggaaaccgttggggacaagattcaggccgtgcagttggaagacggcagacggtttgaaacctgcgcggtggcgtcgaacgctgatgttgtacatacctatcgcgatctgctgtctcagcatcccgcagccgctaagcaggcgaaaaaactgcaatccaagcgtatgagtaactcactgtttgtactctattttggtctcaaccatcatcacgatcaactcgcccatcataccgtctgttttgggccacgctaccgtgaactgattcacgaaatttttaaccatgatggtctggctgaggatttttcgctttatttacacgcaccttgtgtcacggatccgtcactggcaccggaagggtgcggcagctattatgtgctggcgcctgttccacacttaggcacggcgaacctcgactgggcggtagaaggaccccgactgcgcgatcgtatttttgactaccttgagcaacattacatgcctggcttgcgaagccagttggtgacgcaccgtatgtttacgccgttcgatttccgcgacgagctcaatgcctggcaaggttcggccttctcggttgaacctattctgacccagagcgcctggttccgaccacataaccgcgataagcacattgataatctttatctggttggcgcaggcacccatcctggcgcgggcattcccggcgtaatcggctcggcgaaggcgacggcaggcttaatgctggaggacctgatttga

SEQ ID NO: 8 - protein, 492 aa, Pantoea  stewartii
MKPTTVIGAGFGGLALAIRLQAAGIPVLLLEQRDKPGGRAYVYQEQGFTFDAGPTVITDPSAIEELFALAGKQLKDYVELLPVTPFYRLCWESGKVFNYDNDQAQLEAQIQQFNPRDVAGYRAFLDYSRAVFNEGYLKLGTVPFLSFKDMLRAAPQLAKLQAWRSVYSKVAGYIEDEHLRQAFSFHSLLVGGNPFATSSIYTLIHALEREWGVWFPRGGTGALVNGMIKLFQDLGGEVVLNARVSHMETVGDKIQAVQLEDGRRFETCAVASNADVVHTYRDLLSQHPAAAKQAKKLQSKRMSNSLFVLYFGLNHHHDQLAHHTVCFGPRYRELIHEIFNHDGLAEDFSLYLHAPCVTDPSLAPEGCGSYYVLAPVPHLGTANLDWAVEGPRLRDRIFDYLEQHYMPGLRSQLVTHRMFTPFDFRDELNAWQGSAFSVEPILTQSAWFRPHNRDKHIDNLYLVGAGTHPGAGIPGVIGSAKATAGLMLEDLI

SEQ ID NO: 9 - DNA, 891 bp, Pantoea  stewartii
atggcggttggctcgaaaagctttgcgactgcatcgacgcttttcgacgccaaaacccgtcgcagcgtgctgatgctttacgcatggtgccgccactgcgacgacgtcattgacgatcaaacactgggctttcatgccgaccagccctcttcgcagatgcctgagcagcgcctgcagcagcttgaaatgaaaacgcgtcaggcctacgccggttcgcaaatgcacgagcccgcttttgccgcgtttcaggaggtcgcgatggcgcatgatatcgctcccgcctacgcgttcgaccatctggaaggttttgccatggatgtgcgcgaaacgcgctacctgacactggacgatacgctgcgttattgctatcacgtcgccggtgttgtgggcctgatgatggcgcaaattatgggcgttcgcgataacgccacgctcgatcgcgcctgcgatctcgggctggctttccagttgaccaacattgcgcgtgatattgtcgacgatgctcaggtgggccgctgttatctgcctgaaagctggctggaagaggaaggactgacgaaagcgaattatgctgcgccagaaaaccggcaggccttaagccgtatcgccgggcgactggtacgggaagcggaaccctattacgtatcatcaatggccggtctggcacaattacccttacgctcggcctgggccatcgcgacagcgaagcaggtgtaccgtaaaattggcgtgaaagttgaacaggccggtaagcaggcctgggatcatcgccagtccacgtccaccgccgaaaaattaacgcttttgctgacggcatccggtcaggcagttacttcccggatgaagacgtatccaccccgtcctgctcatctctggcagcgcccgatctag

SEQ ID NO: 10 - protein, 296 aa, Pantoea  stewartii
MAVGSKSFATASTLFDAKTRRSVLMLYAWCRHCDDVIDDQTLGFHADQPSSQMPEQRLQQLEMKTRQAYAGSQMHEPAFAAFQEVAMAHDIAPAYAFDHLEGFAMDVRETRYLTLDDTLRYCYHVAGVVGLMMAQIMGVRDNATLDRACDLGLAFQLTNIARDIVDDAQVGRCYLPESWLEEEGLTKANYAAPENRQALSRIAGRLVREAEPYYVSSMAGLAQLPLRSAWAIATAKQVYRKIGVKVEQAGKQAWDHRQSTSTAEKLTLLLTASGQAVTSRMKTYPPRPAHLWQRPI

SEQ ID NO: 11 - DNA, 528 bp, Pantoea  stewartii
atgttgtggatttggaatgccctgatcgtgtttgtcaccgtggtcggcatggaagtggttgctgcactggcacataaatacatcatgcacggctggggttggggctggcatctttcacatcatgaaccgcgtaaaggcgcatttgaagttaacgatctctatgccgtggtattcgccattgtgtcgattgccctgatttacttcggcagtacaggaatctggccgctccagtggattggtgcaggcatgaccgcttatggtttactgtattttatggtccacgacggactggtacaccagcgctggccgttccgctacataccgcgcaaaggctacctgaaacggttatacatggcccaccgtatgcatcatgctgtaaggggaaaagagggctgcgtgtcctttggttttctgtacgcgccaccgttatctaaacttcaggcgacgctgagagaaaggcatgcggctagatcgggcgctgccagagatgagcaggacggggtggatacgtcttcatccgggaagtaa

SEQ ID NO: 12 - protein, 175 aa, Pantoea  stewartii
MLWIWNALIVFVTVVGMEVVAALAHKYIMHGWGWGWHLSHHEPRKGAFEVNDLYAVVFAIVSIALIYFGSTGIWPLQWIGAGMTAYGLLYFMVHDGLVHQRWPFRYIPRKGYLKRLYMAHRMHHAVRGKEGCVSFGFLYAPPLSKLQATLRERHAARSGAARDEQDGVDTSSSGK

SEQ ID NO: 13 - DNA, 25 bp, Artificial Sequence
  misc_feature: Primer
atgacggtctgcgcaaaaaaacacg

SEQ ID NO: 14 - DNA, 28 bp, Artificial Sequence
  misc_feature: Primer
gagaaattatgttgtggatttggaatgc

(19) United States
(12) Patent Application Publication
(10) Pub. No.: US 2003/0148319 A1
(43) Pub. Date: Aug. 7, 2003

(54) Genes encoding carotenoid compounds

(76) Inventors: Patricia C. Brzostowicz, West Chester, PA (US); Qiong Cheng, Wilmington, DE (US); Stephen K. Picataggio, Landenberg, PA (US); Pierre E. Rouviere, Wilmington, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/218,118

(22) Filed: Aug. 13, 2002

Related U.S. Application Data
(60) Provisional application No. 60/312,646, filed on Aug. 15, 2001.

(51) Int. Cl. C12Q 1/68; C12P 23/00; C12N 9/10; C07H 21/04; C12P 21/02; C12N 5/06

(52) U.S. Cl. 435/6; 435/67; 435/69.1; 435/193; 435/320.1; 435/325; 536/23.2

(57) ABSTRACT

Genes have been isolated from Pantoea stewartii encoding geranylgeranyl pyrophosphate (GGPP) synthase (crtE), phytoene synthase (crtB), phytoene desaturase(crtI), lycopene cyclase(crtY), β-carotene hydroxylase(crtZ), and zeaxanthin glucosyl transferase (crtX) activity. The genes and their products are useful for the conversion of phytoene to the carotenoids. Vectors containing those DNA segments, host cells containing the vectors and methods for producing those enzymes and β-carotene by recombinant DNA technology in transformed host organisms are disclosed.

### FIELD OF THE INVENTION

The invention relates to the field of molecular biology and microbiology. More specifically, carotenoid biosynthetic genes have been isolated from Pantoea stewartii and expressed in prokaryotic hosts such as Escherichia coli (E. coli, leading to production of the carotenogenic compounds lycopene, β-carotene, zeaxanthin, and zeaxanthin-β-diglucoside. The present invention also relates to processes for producing such carotenoid compounds.

### BACKGROUND OF THE INVENTION

Carotenoids represent one of the most widely distributed and structurally diverse classes of natural pigments, producing light yellow to orange to deep red color. Eye-catching examples of carotenogenic tissues include carrots, tomatoes, red peppers, and the petals of daffodils and marigolds. Carotenoids are synthesized by all photosynthetic organisms, as well as some bacteria and fungi. These pigments have important functions in photosynthesis, nutrition, and protection against photooxidative damage. For example, animals do not have the ability to synthesize carotenoids but must obtain these nutritionally important compounds through their dietary sources. Structurally, carotenoids are 40-carbon (C₄₀) terpenoids derived from the isoprene biosynthetic pathway and its five-carbon universal isoprene building block, isopentenyl pyrophosphate (IPP).

Typically, the formation of phytoene (7,8,11,12,7′,8′,11′,12′-ω-octahydro-ω, ω-carotene) represents the first step unique to biosynthesis of carotenoids (FIGS. 1 and 2). Phytoene itself is a colorless carotenoid and occurs via isomerization of IPP to dimethylallyl pyrophosphate (DMAPP) by isopentenyl pyrophosphate isomerase. The reaction is followed by a sequence of 3 prenyltransferase reactions in which geranyl pyrophosphate (GPP), farnesyl pyrophosphate (FPP), and geranylgeranyl pyrophosphate (GGPP) are formed. The gene crtE, encoding GGPP synthetase (EC 2.5.1.29), is responsible for this latter reaction. Finally, two molecules of GGPP condense to form phytoene (PPPP), catalyzed by crtB, encoding phytoene synthase (EC 2.5.1.-).

Lycopene is the first “colored” carotenoid produced from phytoene. Lycopene imparts the characteristic red color to ripe tomatoes and has great utility as a food colorant. It is also an intermediate in the biosynthesis of other carotenoids in some bacteria, fungi and green plants. Lycopene is prepared biosynthetically from phytoene through four sequential dehydrogenation reactions by the removal of eight atoms of hydrogen, catalyzed by the gene crtI (encoding phytoene desaturase). Imtermediaries in this reaction are phtyofluene, zeta-carotene, and neurosporene.

Lycopene cyclase (crtY) converts lycopene to β-carotene (β,β-carotene), the second “colored” carotenoid. β-carotene is a typical carotene with a color spectrum ranging from yellow to orange. Its utility is as a colorant for margarine and butter, as a source for vitamin A production, and recently as a compound with potential preventative effects against certain kinds of cancers.

β-carotene is converted to zeaxanthin ((3R,3′R)-β,β-carotene-3,3′-diol) via a hydroxylation reaction resulting from the activity of β-carotene hydroxylase (encoded by the crtZ gene). Zeaxanthin is a xanthophyll with a color spectrum ranging from yellow to orange. For example, it is the yellow pigment which is present in the seeds of maize. Zeaxanthin is contained in feeds for hen or colored carp and is an important pigment source for their coloration. Finally, zeaxanthin can be converted to zeaxanthin-β-diglucoside. This reaction is catalyzed by zeaxanthing glucosyl transferase (EC 2.4.1.-; encoded by the crtX gene).

Several reviews discuss the genetics of carotenoid pigment biosynthesis, such as those of Armstrong (J. Bact. 176: 4795-4802 (1994); Annu. Rev. Microbiol. 51:629-659 (1997)). This pathway is extremely well studied in the Gram-negative, pigmented bacteria of the genera Pantoea, formerly known as Erwinia. In both E. herbicola EHO-10 (ATCC 39368) and E. uredovora 20D3 (ATCC 19321), the crt genes are clustered in two operons, crt Z and crtEXYIB (U.S. Pat. No. 5,656,472; U.S. Pat. No. 5,5545,816; U.S. Pat. No. 5,530,189; U.S. Pat. No. 5,530,188; U.S. Pat. No. 5,429,939). Despite the similarity in operon structure, the DNA sequences of E. uredovora and E. herbicola show no homology by DNA-DNA hybridization (U.S. Pat. No. 5,429,939).

Although more than 600 different carotenoids have been identified in nature, only a few are used industrially for food colors, animal feeding, pharmaceuticals and cosmetics. Presently, most of the carotenoids used for industrial purposes are produced by chemical synthesis; however, these compounds are very difficult to make chemically (Nelis and Leenheer, Appl. Bacteriol. 70:181-191 (1991)). Natural carotenoids can either be obtained by extraction of plant material or by microbial synthesis. At the present time, only a few plants are widely used for commercial carotenoid production. However, the productivity of carotenoid synthesis in these plants is relatively low and the resulting carotenoids are very expensive. One way to increase the productive capacity of biosynthesis would be to apply recombinant DNA technology (reviewed in Misawa and Shimada, J. Biotech. 59:169-181 (1998)). Thus, it would be desirable to produce carotenoids in non-carotenogenic bacteria and yeasts, thereby permitting control over quality, quantity and selection of the most suitable and efficient producer organisms. The latter is especially important for commercial production economics and therefore availability to consumers.

One organism capable of carotenoid synthesis and a potential source of genes for such an endeavor is Pantoea stewartii subsp. stewartii (ATCC No. 8199). The former genus Erwinia has undergone substantial reclassification within the last few decades, following extensive analysis. The current classification of Pantoea ananatis (formerly Erwinia uredovora), Pantoea stewartii subsp. stewartii (formerly Erwinia stewartii), and Pantoea agglomerans (formerly Erwinia herbicola) are described at http://www.bacterio.cict.fr/p/pantoea.html and http://www.bacterio.cict.fr/e/enterobacter.html.

Applicants note that although much of contemporary taxonomy relies heavily on phenotypic characterization for the definition of genera and species, the current nomenclature which clearly recognizes Pantoea ananatis, Pantoea stewartii subsp. stewartii, and Pantoea agglomerans as distinct and separate microorganisms is additionally based on DNA hybridization analyses. Comparison of nucleic acid relatedness, based on the reassociation kinetics of denatured genomic DNA mixtures, provide a more precise means of measuring the relatedness of two organisms.

Although most of the genes involved in the carotenoid biosynthetic pathway are known in Pantoea ananatis and Pantoea agglomerans, the genes involved for Pantoea stewartii subsp. stewartii are not described in the existing literature. The problem to be solved, therefore, is to identify nucleic acid sequences encoding all or a portion of these carotenoid biosynthetic enzymes to facilitate studies to better understand carotenoid biosynthetic pathways, provide genetic tools for the manipulation of those pathways, and provide a means to synthesize carotenoids in large amounts by introducing and expressing the appropriate gene(s) in an appropriate host. This will lead to carotenoid production superior to synthetic methods.

Applicants have solved the stated problem by isolating six unique open reading frames (ORFs) encoding enzymes in the carotenoid biosynthic pathway from Pantoea stewartii subsp. stewartii.

### SUMMARY OF THE INVENTION

The invention provides six genes, isolated from Pantoea stewartii that have been demonstrated to be involved in the synthesis of various carotenoids including lycopene, β-carotene, zeaxanthin, and zeaxanthin-β-diglucoside. The genes are clustered on the same operon and include the crtE, X, Y, I, B and Z genes. The DNA sequences of the crtE, X, Y, I, B and Z correspond to ORF's 1-6 and SEQ ID NOs:1, 3, 5, 7, 9 and 11, respectively.

Accordingly the invention provides an isolated nucleic acid molecule encoding a carotenoid biosynthetic enzyme, selected from the group consisting of:

(a) an isolated nucleic acid molecule encoding the amino acid sequence selected from the group consisting of SEQ ID NOs:2, 4, 6, 8, 10, and 12;

(b) an isolated nucleic acid molecule that hybridizes with (a) under the following hybridization conditions: 0.1×SSC, 0.1% SDS, 65° C. and washed with 2×SSC, 0.1% SDS followed by 0.1×SSC, 0.1% SDS; and

(c) an isolated nucleic acid molecule that is complementary to (a) or (b).

The invention additionally provides polypeptides encoded by the instant genes and genetic chimera comprising suitable regulatory regions for genetic expression of the genes in plants or microbes, as well as transformed host comprising the same.

The invention provides a method of obtaining a nucleic acid molecule encoding a carotenoid biosynthetic enzyme comprising:

(a) probing a genomic library with the present nucleic acid molecules;

(b) identifying a DNA clone that hybridizes with the present nucleic acid molecules; and

(c) sequencing the genomic fragment that comprises the clone identified in step (b),

wherein the sequenced genomic fragment encodes a carotenoid biosynthetic enzyme.

Similarly the invention provides a method of obtaining a nucleic acid molecule encoding a carotenoid biosynthetic enzyme comprising:

(a) synthesizing at least one oligonucleotide primer corresponding to a portion of the present nucleic acid sequences; and

(b) amplifying an insert present in a cloning vector using the oligonucleotide primer of step (a);

wherein the amplified insert encodes a portion of an amino acid sequence encoding a carotenoid biosynthetic enzyme.

In a preferred embodiment the invention provides a method for the production of carotenoid compounds comprising:

(a) providing a transformed host cell comprising:

(i) suitable levels of isopentenyl pyrophosphate; and

(ii) a set of nucleic acid molecules encoding the present carotenoid enzymes under the control of suitable regulatory sequences;

(b) contacting the host cell of step (a) under suitable growth conditions with an effective amount of a fermentable carbon substrate whereby a carotenoid compound is produced.

Additionally the invention provides a method of regulating carotenoid biosynthesis in an organism comprising, over-expressing at least one carotenoid gene selected from the group consisting of SEQ ID NOs:1, 3, 5, 7, 9, and 11 in an organism such that the carotenoid biosynthesis is altered in the organism.

In an alternate embodiment the invention provides a mutated gene encoding a carotenoid enzyme having an altered biological activity produced by a method comprising the steps of:

(i) digesting a mixture of nucleotide sequences with restriction endonucleases wherein said mixture comprises:

a) an isolated nucleic acid molecule encoding a carotenoid biosynthetic enzyme selected from the group consisting of SEQ ID NOs: 1, 3, 5, 7, 9, and 11;

b) a first population of nucleotide fragments which will hybridize to said isolated nucleic acid molecules of step (a);

c) a second population of nucleotide fragments which will not hybridize to said isolated nucleic acid molecules of step (a);

wherein a mixture of restriction fragments are produced;

(ii) denaturing said mixture of restriction fragments;

(iii) incubating the denatured said mixture of restriction fragments of step (ii) with a polymerase;

(iv) repeating steps (ii) and (iii) wherein a mutated carotenoid gene is produced encoding a protein having an altered biological activity.

### BRIEF DESCRIPTION OF THE DRAWINGS AND SEQUENCE DESCRIPTIONS

FIG. 1 shows the carotenoid biosynthetic pathway.

FIG. 2 shows the chemical structures involved in the present carotenoid pathway.

FIG. 3 shows one gene cluster containing the carotenoid biosynthetic genes crtEXYIB. The invention can be more fully understood from the following detailed description and the accompanying sequence descriptions which form a part of this application.

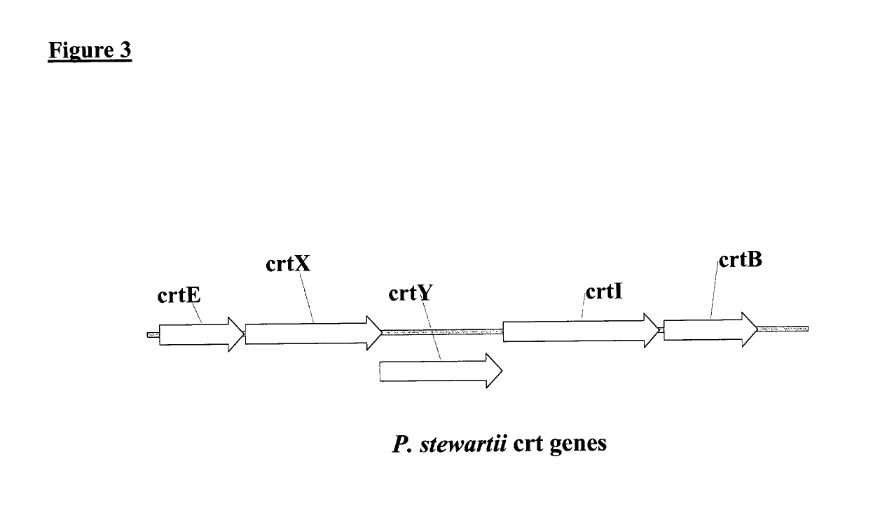

The following sequences are in conformity with 37 C.F.R. 1.821-1.825 (“Requirements for Patent Applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NOs:1-12 are full length genes or proteins as identified in Table 1.

TABLE 1
Summary of Gene and Protein SEQ ID Numbers

| | | Nucleic acid | Peptide |
|---|---|---|---|
| Description | ORF No. | SEQ ID NO. | SEQ ID NO. |
| crtE | 1 | 1 | 2 |
| crtX | 2 | 3 | 4 |
| crtY | 3 | 5 | 6 |
| crtI | 4 | 7 | 8 |
| crtB | 5 | 9 | 10 |
| crtZ | 6 | 11 | 12 |

SEQ ID NOs: 13 and 14 are oligonucleotide primers used to amplify the carotenoid biosynthetic gene cluster from Pantoea stewartii.

### DETAILED DESCRIPTION OF THE INVENTION

The genes and their expression products are useful for the creation of recombinant organisms that have the ability to produce various carotenoid compounds. Nucleic acid fragments encoding the above mentioned enzymes have been isolated from a strain of Pantoea stewartii subsp. stewartii and identified by comparison to public databases containing nucleotide and protein sequences using the BLAST and FASTA algorithms well known to those skilled in the art. The genes and gene products of the present invention may be used in a variety of ways for the enhancement or manipulation of carotenoid compounds. There is a general practical utility for microbial production of carotenoid compounds as these compounds are very difficult to make chemically (Nelis and Leenheer, Appl. Bacteriol. 70:181-191 (1991)). Most carotenoids have strong color and can be viewed as natural pigments or colorants. Furthermore, many carotenoids have potent antioxidant properties and thus inclusion of these compounds in the diet is thought to be healthful. Well-known examples are β-carotene and astaxanthin. Additionally, carotenoids are required elements of aquaculture. Salmon and shrimp aquacultures are particularly useful applications for this invention as carotenoid pigmentation is critically important for the value of these organisms. (Shahidi, F., and J. A. Brown, Critical reviews in Food Science 38(1): 1-67 (1998)). Finally, carotenoids have utility as intermediates in the synthesis of steroids, flavors and fragrances and compounds with potential electro-optic applications.

The disclosure below provides a detailed description of the isolation of carotenoid synthesis genes from Pantoea stewartii subsp. stewartii, modification of these genes by genetic engineering, and their insertion into compatible plasmids suitable for cloning and expression in E. coli yeasts, fungi and higher plants. Also disclosed are methods for preparation of the appropriate enzymes and the methods for β-carotene production in these various hosts.

Definitions

In this disclosure, a number of terms and abbreviations are used. The following definitions are provided.

“Open reading frame” is abbreviated ORF.

“Polymerase chain reaction” is abbreviated PCR.

“High Performance Liquid Chromatography” is abbreviated HPLC.

The term “Pantoea agglomerans” is used interchangeably with the name Erwinia herbicola (Beji et al., Int. J. Syst. Bacteriol. 38:77-88 (1988); Gavini et al., Int. J. Syst. Bacteriol. 39:337-345 (1989)).

The term “Pantoea ananatis” is used interchangeably with the name Erwinia uredovora (Mergaert et al., Int. J. Syst. Bacteriol. 43:162-173 (1993)).

The term “Pantoea stewartii subsp. stewartii” is abbreviated as “Pantoea stewartii” and is used interchangeably with Erwinia stewartii (Mergaert et al., supra).

The term “carotenoid” means any lipophilic isoprenoid compound, produced either synthetically or naturally. All carotenoids possess molecules of isopentenyl pyrophosphate (IPP) as the universal isoprene building block.

The term “CrtE” refers to the geranylgeranyl pyrophosphate synthase enzyme encoded by the crtE gene represented in SEQ ID NO:1, and which converts trans-trans-farnesyl diphosphate and isopentenyl diphosphate to pyrophosphate and geranylgeranyl diphosphate.

The term “CrtX” refers to the zeaxanthin glucosyl transferase enzyme encoded by the crtX gene represented in SEQ ID NO:3, and which converts to zeaxanthin to zeaxanthin-β-diglucoside.

The term “CrtY” refers to the lycopene cyclase enzyme encoded by the crtY gene represented in SEQ ID NO:5, which converts lycopene to β-carotene.

The term “CrtI” refers to the phytoene dehydrogenase enzyme encoded by the crtI gene represented in SEQ ID NO:7. CrtI converts phytoene into lycopene via the intermediaries of phytofluene, zeta-carotene and neurosporene by the introduction of 4 double bonds.

The term “CrtB” refers to the phytoene synthase enzyme encoded by the crtB gene represented in SEQ ID NO:9, which catalyzes the reaction from prephytoene diphosphate to phytoene.

The term “CrtZ” refers to the lycopene cyclase enzyme encoded by the crtZ gene represented in SEQ ID NO:11, which catalyzes a hydroxylation reaction from β-carotene to zeaxanthin.

The term “carotenoid biosynthetic enzyme” is an inclusive term referring to any and all of the enzymes in the present pathway including CrtE, CrtX, CrtY, CrtI, CrtB, and CrtZ.

As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

As used herein, “substantially similar” refers to nucleic acid fragments wherein changes in one or more nucleotide bases result in substitution of one or more amino acids, but do not affect the functional properties of the protein encoded by the DNA sequence. “Substantially similar” also refers to nucleic acid fragments wherein changes in one or more nucleotide bases do not affect the ability of the nucleic acid fragment to mediate alteration of gene expression by antisense or co-suppression technology. “Substantially similar” also refers to modifications of the nucleic acid fragments of the instant invention, such as deletion or insertion of one or more nucleotide bases that do not substantially affect the functional properties of the resulting transcript. It is therefore understood that the invention encompasses more than the specific exemplary sequences.

For example, it is well known in the art that alterations in a gene which result in the production of a chemically equivalent amino acid at a given site, but do not effect the functional properties of the encoded protein are common. For the purposes of the present invention substitutions are defined as exchanges within one of the following five groups:

1. Small aliphatic, nonpolar or slightly polar residues: Ala, Ser, Thr (Pro, Gly);

2. Polar, negatively charged residues and their amides: Asp, Asn, Glu, Gin;

3. Polar, positively charged residues: His, Arg, Lys;

4. Large aliphatic, nonpolar residues: Met, Leu, lie, Val (Cys); and

5. Large aromatic residues: Phe, Tyr, Trp.

Thus, a codon for the amino acid alanine, a hydrophobic amino acid, may be substituted by a codon encoding another less hydrophobic residue (such as glycine) or a more hydrophobic residue (such as valine, leucine, or isoleucine). Similarly, changes which result in substitution of one negatively charged residue for another (such as aspartic acid for glutamic acid) or one positively charged residue for another (such as lysine for arginine) can also be expected to produce a functionally equivalent product.

In many cases, nucleotide changes which result in alteration of the N-terminal and C-terminal portions of the protein molecule would also not be expected to alter the activity of the protein.

Each of the proposed modifications is well within the routine skill in the art, as is determination of retention of biological activity of the encoded products. Moreover, the skilled artisan recognizes that substantially similar sequences encompassed by this invention are also defined by their ability to hybridize, under stringent conditions (0.1×SSC, 0.1% SDS, 65° C. and washed with 2×SSC, 0.1% SDS followed by 0.1×SSC, 0.1% SDS), with the sequences exemplified herein. Preferred substantially similar nucleic acid fragments of the instant invention are those nucleic acid fragments whose DNA sequences are at least 80% identical to the DNA sequence of the nucleic acid fragments reported herein. More preferred nucleic acid fragments are at least 90% identical to the DNA sequence of the nucleic acid fragments reported herein. Most preferred are nucleic acid fragments that are at least 95% identical to the DNA sequence of the nucleic acid fragments reported herein.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above, except the temperature of the final two 30 min washes in 0.2×SSC, 0.5% SDS was increased to 60° C. Another preferred set of highly stringent conditions uses two final washes in 0.1×SSC, 0.1% SDS at 65° C. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferable a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

A “substantial portion” of an amino acid or nucleotide sequence is that portion comprising enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Basic Local Alignment Search Tool; Altschul, S. F., et al., (1993) J. Mol. Biol. 215:403-410; see also www.ncbi.nlm.nih.gov/BLAST/). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence. The instant specification teaches partial or complete amino acid and nucleotide sequences encoding one or more particular microbial proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

The term “complementary” is used to describe the relationship between nucleotide bases that are capable of hybridizing to one another. For example, with respect to DNA, adenosine is complementary to thymine and cytosine is complementary to guanine. Accordingly, the instant invention also includes isolated nucleic acid fragments that are complementary to the complete sequences as reported in the accompanying Sequence Listing, as well as those substantially similar nucleic acid sequences.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

Suitable nucleic acid fragments (isolated polynucleotides of the present invention) encode polypeptides that are at least about 70% identical, preferably at least about 80% identical to the amino acid sequences reported herein. Preferred nucleic acid fragments encode amino acid sequences that are about 85% identical to the amino acid sequences reported herein. More preferred nucleic acid fragments encode amino acid sequences that are at least about 90% identical to the amino acid sequences reported herein. Most preferred are nucleic acid fragments that encode amino acid sequences that are at least about 95% identical to the amino acid sequences reported herein. Suitable nucleic acid fragments not only have the above homologies but typically encode a polypeptide having at least 50 amino acids, preferably at least 100 amino acids, more preferably at least 150 amino acids, still more preferably at least 200 amino acids, and most preferably at least 250 amino acids.

“Codon degeneracy” refers to the nature in the genetic code permitting variation of the nucleotide sequence without effecting the amino acid sequence of an encoded polypeptide. Accordingly, the instant invention relates to any nucleic acid fragment that encodes all or a substantial portion of the amino acid sequence encoding the instant microbial polypeptides as set forth in SEQ ID NOs: 2, 4, 6, 8, 10, and 12. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector binding sites and stem-loop structures.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into protein by the cell. “cDNA” refers to a double-stranded DNA that is complementary to and derived from mRNA. “Sense” RNA refers to RNA transcript that includes the mRNA and so can be translated into protein by the cell.

“Antisense RNA” refers to a RNA transcript that is complementary to all or part of a target primary transcript or mRNA and that blocks the expression of a target gene (U.S. Pat. No. 5,107,065; WO 9928508). The complementarity of an antisense RNA may be with any part of the specific gene transcript, i.e., at the 5′ non-coding sequence, 3′ non-coding sequence, or the coding sequence. “Functional RNA” refers to antisense RNA, ribozyme RNA, or other RNA that is not translated yet has an effect on cellular processes.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. Expression may also refer to translation of mRNA into a polypeptide.

“Mature” protein refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propeptides may be but are not limited to intracellular localization signals.

The term “signal peptide” refers to an amino terminal polypeptide preceding the secreted mature protein. The signal peptide is cleaved from, and is therefore not present in, the mature protein. Signal peptides have the function of directing and translocating secreted proteins across cell membranes. A signal peptide is also referred to as a signal protein.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The term “fermentable carbon substrate” refers to a carbon source capable of being metabolized by host organisms of the present invention and particularly carbon sources selected from the group consisting of monosaccharides, oligosaccharides, polysaccharides, and one-carbon substrates or mixtures thereof.

The terms “plasmid”, “vector” and “cassette” refer to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

The term “altered biological activity” will refer to an activity, associated with a protein encoded by a microbial nucleotide sequence which can be measured by an assay method, where that activity is either greater than or less than the activity associated with the native microbial sequence. “Enhanced biological activity” refers to an altered activity that is greater than that associated with the native sequence. “Diminished biological activity” is an altered activity that is less than that associated with the native sequence.

The term “sequence analysis software” refers to any computer algorithm or software program that is useful for the analysis of nucleotide or amino acid sequences. “Sequence analysis software” may be commercially available or independently developed. Typical sequence analysis software will include, but is not limited to: the GCG suite of programs (Wisconsin Package Version 9.0, Genetics Computer Group (GCG), Madison, Wis.), BLASTP, BLASTN, BLASTX (Altschul et al., J. Mol. Biol. 215:403-410 (1990)), and DNASTAR (DNASTAR, Inc., St. Madison, Wis.), and the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Publisher: Plenum, New York, N.Y.). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized.

Standard recombinant DNA and molecular cloning techniques used herein are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et al., Current Protocols in Molecular Biology, published by Greene Publishing Assoc. and Wiley-Interscience (1987).

A variety of nucleotide sequences have been isolated from Pantoea stewartii encoding gene products involved in carotenoid production. For example, crt genes E, X, Y, I, B and Z which lead to the production of the pigmented carotenoids lycopene and β-carotene have been isolated.

Comparison of the crtE nucleotide base and deduced amino acid sequences to public databases reveals that the most similar known sequences range from about 83% identical to the amino acid sequence of crtE reported herein over length of 303 amino acids using a Smith-Waterman alignment algorithm (W. R. Pearson, Comput. Methods Genome Res., [Proc. Int. Symp.] (1994), Meeting Date 1992, 111-20. Editor(s): Suhai, Sandor. Publisher: Plenum, New York, N.Y.). More preferred amino acid fragments are at least about 80%-90% identical to the sequences herein. Most preferred are nucleic acid fragments that are at least 95% identical to the amino acid fragments reported herein. Similarly, preferred crtE encoding nucleic acid sequences corresponding to the instant ORF's are those encoding active proteins and which are at least 80% identical to the nucleic acid sequences of reported herein. More preferred crtE nucleic acid fragments are at least 90% identical to the sequences herein. Most preferred are crtE nucleic acid fragments that are at least 95% identical to the nucleic acid fragments reported herein.

Comparison of the crtX nucleotide base and deduced amino acid sequences to public databases reveals that the most similar known sequences range from about 75% identical to the amino acid sequence of crtX reported herein over length of 431 amino acids using a Smith-Waterman alignment algorithm (W. R. Pearson, supra). More preferred amino acid fragments are at least about 80%-90% identical to the sequences herein. Most preferred are nucleic acid fragments that are at least 95% identical to the amino acid fragments reported herein. Similarly, preferred crtX encoding nucleic acid sequences corresponding to the instant ORF's are those encoding active proteins and which are at least 80% identical to the nucleic acid sequences of reported herein. More preferred crtX nucleic acid fragments are at least 90% identical to the sequences herein. Most preferred are crtX nucleic acid fragments that are at least 95% identical to the nucleic acid fragments reported herein.

Comparison of the crtY nucleotide base and deduced amino acid sequences to public databases reveals that the most similar known sequences range from about 83% identical to the amino acid sequence of crtY reported herein over length of 382 amino acids using a Smith-Waterman alignment algorithm (W. R. Pearson, supra). More preferred amino acid fragments are at least about 80%-90% identical to the sequences herein. Most preferred are nucleic acid fragments that are at least 95% identical to the amino acid fragments reported herein. Similarly, preferred crtY encoding nucleic acid sequences corresponding to the instant ORF's are those encoding active proteins and which are at least 80% identical to the nucleic acid sequences of reported herein. More preferred crtY nucleic acid fragments are at least 90% identical to the sequences herein. Most preferred are crtY nucleic acid fragments that are at least 95% identical to the nucleic acid fragments reported herein.

Comparison of the crtI nucleotide base and deduced amino acid sequences to public databases reveals that the most similar known sequences range from about 89% identical to the amino acid sequence of crtI reported herein over length of 492 amino acids using a Smith-Waterman alignment algorithm (W. R. Pearson, supra). More preferred amino acid fragments are at least about 80%-90% identical to the sequences herein. Most preferred are nucleic acid fragments that are at least 95% identical to the amino acid fragments reported herein. Similarly, preferred crtI encoding nucleic acid sequences corresponding to the instant ORF's are those encoding active proteins and which are at least 80% identical to the nucleic acid sequences of reported herein. More preferred crtI nucleic acid fragments are at least 90% identical to the sequences herein. Most preferred are crtI nucleic acid fragments that are at least 95% identical to the nucleic acid fragments reported herein.

Comparison of the crtB nucleotide base and deduced amino acid sequences to public databases reveals that the most similar known sequences range from about 88% identical to the amino acid sequence of crtB reported herein over length of 296 amino acids using a Smith-Waterman alignment algorithm (W. R. Pearson, supra). More preferred amino acid fragments are at least about 80%-90% identical to the sequences herein. Most preferred are nucleic acid fragments that are at least 95% identical to the amino acid fragments reported herein. Similarly, preferred crtB encoding nucleic acid sequences corresponding to the instant ORF's are those encoding active proteins and which are at least 80% identical to the nucleic acid sequences of reported herein. More preferred crtB nucleic acid fragments are at least 90% identical to the sequences herein. Most preferred are crtB nucleic acid fragments that are at least 95% identical to the nucleic acid fragments reported herein.

Comparison of the crtZ nucleotide base and deduced amino acid sequences to public databases reveals that the most similar known sequences range from about 88% identical to the amino acid sequence of crtZ reported herein over length of 175 amino acids using a Smith-Waterman alignment algorithm (W. R. Pearson, supra). More preferred amino acid fragments are at least about 80%-90% identical to the sequences herein. Most preferred are nucleic acid fragments that are at least 95% identical to the amino acid fragments reported herein. Similarly, preferred crtZ encoding nucleic acid sequences corresponding to the instant ORF's are those encoding active proteins and which are at least 80% identical to the nucleic acid sequences of reported herein. More preferred crtZ nucleic acid fragments are at least 90% identical to the sequences herein. Most preferred are crtZ nucleic acid fragments that are at least 95% identical to the nucleic acid fragments reported herein.

Isolation of Homologs

The nucleic acid fragments of the instant invention may be used to isolate genes encoding homologous proteins from the same or other microbial species. Isolation of homologous genes using sequence-dependent protocols is well known in the art. Examples of sequence-dependent protocols include, but are not limited to: methods of nucleic acid hybridization; and methods of DNA and RNA amplification, as exemplified by various uses of nucleic acid amplification technologies [e.g. polymerase chain reaction (PCR; Mullis et al., U.S. Pat. No. 4,683,202); ligase chain reaction (LCR; Tabor, S. et al., Proc. Acad. Sci. USA 82: 1074 (1985)); or strand displacement amplification (SDA; Walker, et al., Proc. Natl. Acad. Sci. U.S.A., 89: 392 (1992))].

For example, genes encoding similar proteins or polypetides to those of the instant invention could be isolated directly by using all or a portion of the instant nucleic acid fragments as DNA hybridization probes to screen libraries from any desired bacteria using methodology well known to those skilled in the art. Specific oligonucleotide probes based upon the instant nucleic acid sequences can be designed and synthesized by methods known in the art (Maniatis, supra). Moreover, the entire sequences can be used directly to synthesize DNA probes by methods known to the skilled artisan such as random primers DNA labeling, nick translation, or end-labeling techniques, or RNA probes using available in vitro transcription systems. In addition, specific primers can be designed and used to amplify a part of, or the full-length of, the instant sequences. The resulting amplification products can be labeled directly during amplification reactions or labeled after amplification reactions, and used as probes to isolate full length DNA fragments under conditions of appropriate stringency.

Typically, in PCR-type amplification techniques, the primers have different sequences and are not complementary to each other. Depending on the desired test conditions, the sequences of the primers should be designed to provide for both efficient and faithful replication of the target nucleic acid. Methods of PCR primer design are common and well known in the art (Thein and Wallace, “The use of oligonucleotide as specific hybridization probes in the Diagnosis of Genetic Disorders”, in Human Genetic Diseases: A Practical Approach, K. E. Davis Ed., (1986) pp. 33-50 IRL Press, Herndon, Va.); Rychlik, W. (1993) In White, B. A. (ed.), Methods in Molecular Biology, Vol. 15, pages 31-39, PCR Protocols: Current Methods and Applications. Humania Press, Inc., Totowa, N.J.).

Generally two short segments of the instant sequences may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding microbial genes.

Alternatively, the second primer sequence may be based upon sequences derived from the cloning vector. For example, the skilled artisan can follow the RACE protocol (Frohman et al., PNAS USA 85:8998 (1988)) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara et al., PNAS USA 86:5673 (1989); Loh et al., Science 243:217 (1989)).

Alternatively the instant sequences may be employed as hybridization reagents for the identification of homologs. The basic components of a nucleic acid hybridization test include a probe, a sample suspected of containing the gene or gene fragment of interest, and a specific hybridization method. Probes of the present invention are typically single stranded nucleic acid sequences which are complementary to the nucleic acid sequences to be detected. Probes are “hybridizable” to the nucleic acid sequence to be detected. The probe length can vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base.

Hybridization methods are well defined. Typically the probe and sample must be mixed under conditions which will permit nucleic acid hybridization. This involves contacting the probe and sample in the presence of an inorganic or organic salt under the proper concentration and temperature conditions. The probe and sample nucleic acids must be in contact for a long enough time that any possible hybridization between the probe and sample nucleic acid may occur. The concentration of probe or target in the mixture will determine the time necessary for hybridization to occur. The higher the probe or target concentration the shorter the hybridization incubation time needed. Optionally a chaotropic agent may be added. The chaotropic agent stabilizes nucleic acids by inhibiting nuclease activity. Furthermore, the chaotropic agent allows sensitive and stringent hybridization of short oligonucleotide probes at room temperature [Van Ness and Chen (1991) Nucl. Acids Res. 19:5143-5151]. Suitable chaotropic agents include guanidinium chloride, guanidinium thiocyanate, sodium thiocyanate, lithium tetrachloroacetate, sodium perchlorate, rubidium tetrachloroacetate, potassium iodide, and cesium trifluoroacetate, among others. Typically, the chaotropic agent will be present at a final concentration of about 3M. If desired, one can add formamide to the hybridization mixture, typically 30-50% (v/v).

Various hybridization solutions can be employed. Typically, these comprise from about 20 to 60% volume, preferably 30%, of a polar organic solvent. A common hybridization solution employs about 30-50% v/v formamide, about 0.15 to 1 M sodium chloride, about 0.05 to 0.1 M buffers, such as sodium citrate, Tris-HCl, PIPES or HEPES (pH range about 6-9), about 0.05 to 0.2% detergent, such as sodium dodecylsulfate, or between 0.5-20 mM EDTA, FICOLL (Pharmacia Inc.) (about 300-500 kilodaltons), polyvinylpyrrolidone (about 250-500 kdal), and serum albumin. Also included in the typical hybridization solution will be unlabeled carrier nucleic acids from about 0.1 to 5 mg/mL, fragmented nucleic DNA, e.g., calf thymus or salmon sperm DNA, or yeast RNA, and optionally from about 0.5 to 2% wt/vol glycine. Other additives may also be included, such as volume exclusion agents which include a variety of polar water-soluble or swellable agents (e.g., polyethylene glycol), anionic polymers (e.g., polyacrylate or polymethylacrylate), and anionic saccharidic polymers (e.g., dextran sulfate).

Nucleic acid hybridization is adaptable to a variety of assay formats. One of the most suitable is the sandwich assay format. The sandwich assay is particularly adaptable to hybridization under non-denaturing conditions. A primary component of a sandwich-type assay is a solid support. The solid support has adsorbed to it or covalently coupled to it immobilized nucleic acid probe that is unlabeled and complementary to one portion of the sequence.

Recombinant Expression—Microbial

The genes and gene products of the instant sequences may be produced in heterologous host cells, particularly in the cells of microbial hosts. Expression in recombinant microbial hosts may be useful for the expression of various pathway intermediates, and/or for the modulation of pathways already existing in the host for the synthesis of new products heretofore not possible using the host.

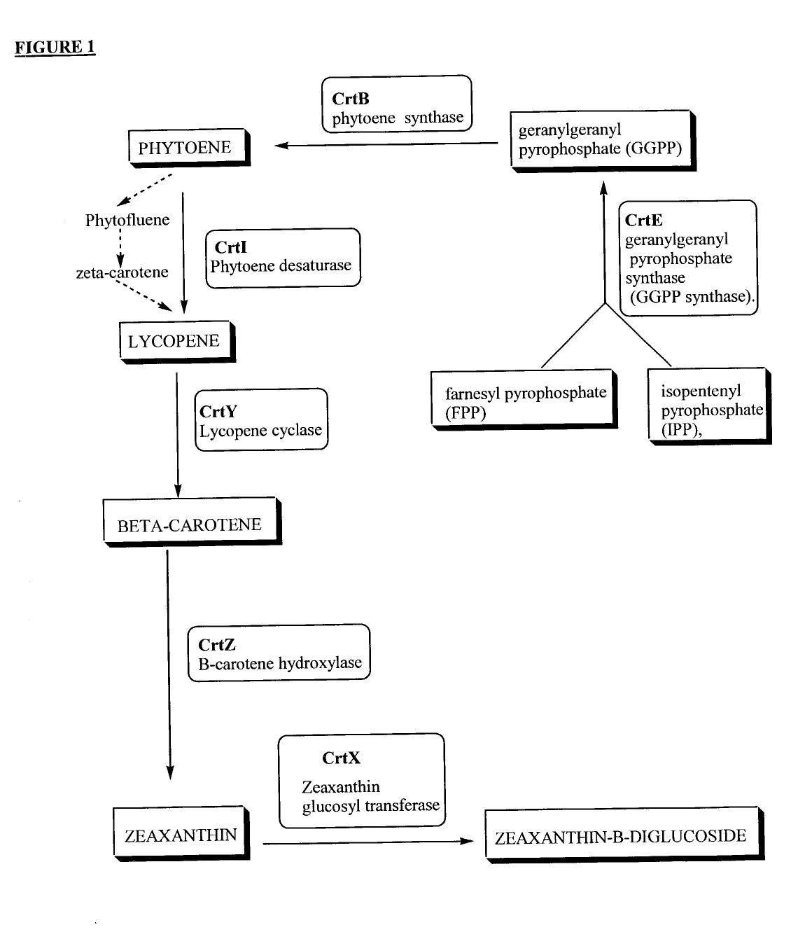

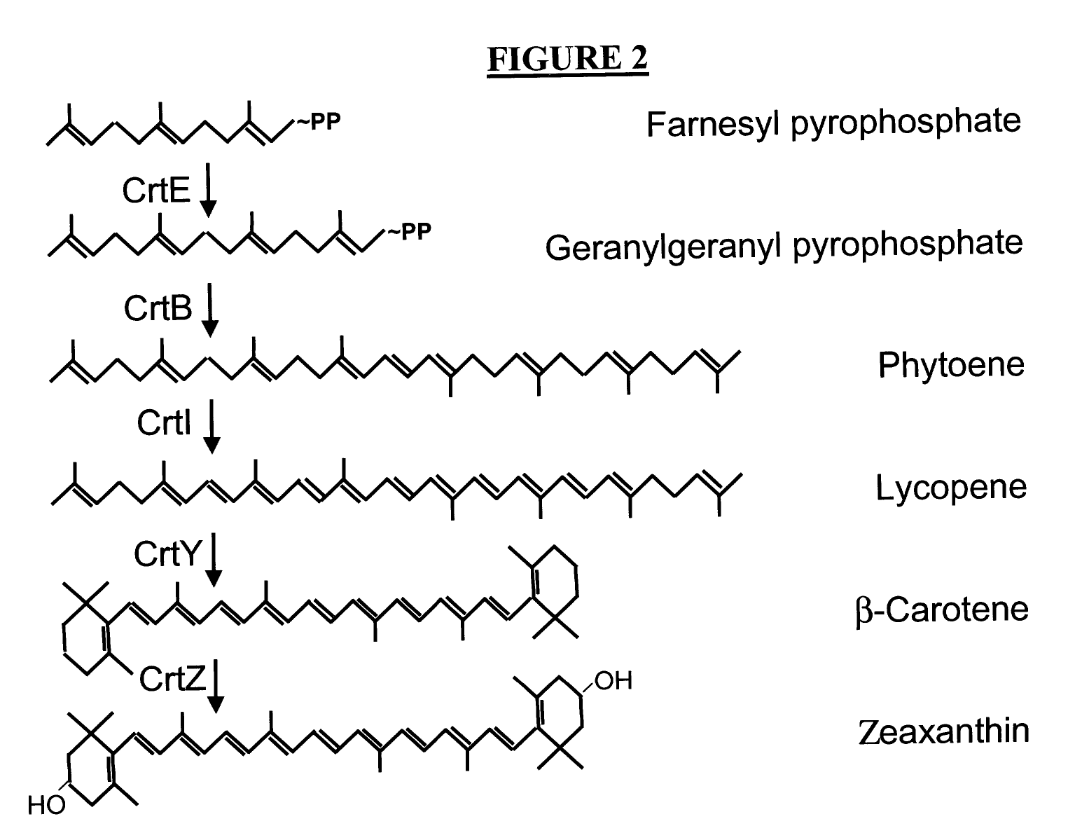

The present genes are particularly useful for the synthesis of carotenoids in organisms that have endogenouse levels of isopentenyl pyrophosphate. Mevalonic acid, the first specific precursor of all the terpenoids is formed from acetyl-CoA via HMG-CoA (3-hydroxy-3-methylglutaryl-CoA), and is itself converted to isopentenyl pyrophosphate (IPP), the universal isoprene unit. After isomerization of IPP to dimethylallyl pyrophosphate and a series of condensation reactions adding IPP, catalyzed by prenyltransferases, geranylgeranyl pyrophosphate (GGPP) is formed according to the scheme in FIGS. 1 and 2. The formation of GGPP is the first step in carotenoid biosynthesis.

In the bacterium Pantoea stewartii(ATCC No. 8199), phytoene has been found to be formed biosynthetically in a two-step process as shown in FIG. 1. The initial step is the condensation of farnesyl pyrophosphate (FPP) and isopentenyl pyrophosphate (IPP) to form geranylgeranyl pyrophosphate (GGPP). This reaction is catalyzed by the enzyme geranylgeranyl pyrophosphate synthase (GGPP synthase). This first step is immediately followed by a tail to tail dimerization of GGPP, catalyzed by the enzyme phytoene synthase, to form phytoene.

Lycopene, which has now been found to be the second carotenoid produced in Pantoea herbicola, is produced from phytoene by the catalytic action of phytoene dehydrogenase-4H. Hence, the carotenoid-specific genes necessary for the synthesis of lycopene from farnesyl pyrophosphate include GGPP synthase, phytoene synthase, and phytoene dehydrogenase-4H.

The third carotenoid produced by Pantoea herbicola results from the cyclization of lycopene to form α-carotene. Little is known about the reaction(s) involved in the cyclization of lycopene (Bramley et al. Current Topics in Cellular Regulation, 29:291-297 (1988)). In the system of the present invention, it is clear that only one enzyme is involved. This enzyme is lycopene cyclase. Thus, the genes required for α-carotene production from farnesyl pyrophosphate include the above-named enzymes/genes plus the gene for lycopene cyclase.

Given this understanding of the relationship between the crt genes, it will be possible to select appropriate microbial host cells for their expression.

Preferred heterologous host cells for expression of the instant genes and nucleic acid fragments are microbial hosts that can be found broadly within the fungal or bacterial families and which grow over a wide range of temperature, pH values, and solvent tolerances. For example, it is contemplated that any bacteria, yeast, and filamentous fungi will be suitable hosts for expression of the present nucleic acid fragments. Because transcription, translation and the protein biosynthetic apparatus are the same irrespective of the cellular feedstock, functional genes are expressed irrespective of carbon feedstock used to generate cellular biomass. Large-scale microbial growth and functional gene expression may utilize a wide range of simple or complex carbohydrates, organic acids and alcohols, and saturated hydrocarbons such as methane or carbon dioxide in the case of photosynthetic or chemoautotrophic hosts. However, the functional genes may be regulated, repressed or depressed by specific growth conditions, which may include the form and amount of nitrogen, phosphorous, sulfur, oxygen, carbon or any trace micronutrient including small inorganic ions. In addition, the regulation of functional genes may be achieved by the presence or absence of specific regulatory molecules that are added to the culture and are not typically considered nutrient or energy sources. Growth rate may also be an important regulatory factor in gene expression. Examples of host strains include, but are not limited to: fungal or yeast species such as Aspergillus, Trichoderma, Saccharomyces, Pichia, Candida, Hansenula, Yarrowia, Rhodosporidium, and Lipomyces; or bacterial species such as Salmonella, Bacillus, Acinetobacter, Zymomonas, Agrobacterium, Flavobacterium, Rhodobacter, Rhodococcus, Streptomyces, Brevibacterium, Corynebacteria, Mycobacterium, Escherichia, Pantoea, Pseudomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium and Klebsiella.

Microbial expression systems and expression vectors containing regulatory sequences that direct high level expression of foreign proteins are well known to those skilled in the art. Any of these could be used to construct chimeric genes for production of any of the gene products of the instant sequences. These chimeric genes could then be introduced into appropriate microorganisms via transformation to provide high level expression of the enzymes.

Accordingly it is expected, for example, that introduction of chimeric genes encoding the instant bacterial enzymes (SEQ ID NO: 2, 4, 6, 8, 10, and 12) under the control of the appropriate promoters will demonstrate increased production of the carotenoids phytoene, lycopene, and/or β-carotene. It is additionally expected that introduction of chimeric genes encoding one or more of the instant sequences will lead to production of carotenoid compounds in the host microbe of choice. Basis for this expectation is found in the ubiquity of the isoprene biosynthetic pathway in microbes. With an appropriate genetic transformation system, it should be possible to genetically engineer a variety of non-carotenogenic hosts. This has been shown, for example, using E. herbicola crt genes, to produce various carotenoids in the hosts E. coli, Agrobacterium tumefaciens, Saccharomyces cerevisiae, Pichia pastoris (yeast), Aspergillus nidulans (fungi), Rhodobacter sphaeroides, and higher plants (U.S. Pat. No. 5,656,472).

It will be appreciated that for the present crt genes to be effective in the production of carotenoids it will be necessary for the host cell to have suitable levels of isopentenyl pyrophosphate (IPP) within the cell. IPP may be supplied exogenously, or may be produced endogenously by the cell, either through native or introduced genetic pathways.

IPP may be synthesized through the well-known acetate/mevalonate pathway; however, recent studies have demonstrated that the mevalonate-dependent pathway does not operate in all living organisms. An alternate mevalonate-independent pathway for IPP biosynthesis has been characterized in bacteria and in green algae and higher plants (Horbach et al., FEMS Microbiol. Lett. 111:135-140 (1993); Rohmer et al, Biochem. 295: 517-524 (1993); Schwender et al., Biochem. 316: 73-80 (1996); Eisenreich et al., Proc. Natl. Acad. Sci. USA 93: 6431-6436 (1996)).

Many steps of isoprenoid pathways are known. For example, the initial steps of the alternate pathway involve the condensation of the 3-carbon molecules pyruvate and D-glyceraldehyde 3-Phosphate to yield a 5-carbon compound of D-1-deoxyxylulose-5-phosphate. This reaction is catalyzed by the dxs gene that encodes D-1-deoxyxylulose-5-phosphate synthase (DXS). This gene's activity has been reported in Mycobacterium tuberculosis (Cole et al., Nature, 393:537-544, 1998).

Next, the isomerization and reduction of D-1-deoxyxylulose-5-phosphate yields 2-C-methyl-D-erythritol-4-phosphate. One of the enzymes involved in the isomerization and reduction process is D-1-deoxyxylulose-5-phosphate reductoisomerase (DXR). The gene product of dxr that catalyzes the formation of 2-C-methyl-D-erythritol-4-phosphate in the alternate pathway has been reported in Mycobacterium tuberculosis (Cole et al., supra).

Steps converting 2-C-methyl-D-erythritol-4-phosphate to isopentenyl monophosphate are not well characterized, although some steps are known. 2-C-methyl-D-erythritol-4-phosphate is then converted into 4-diphosphocytidyl-2C-methyl-D-erythritol in a CTP dependent reaction by the enzyme encoded by the non-annotated gene ygbP. Cole et al. (supra) reported a YgbP protein in Mycobacterium tuberculosis that catalyzes the reaction mentioned above. Recently, the ygbP gene was renamed as ispD as a part of an isp gene cluster (SwissProt #Q46893).

Then the 2^{nd} position hydroxy group of 4-diphosphocytidyl-2C-methyl-D-erythritol can be phosphorylated in an ATP dependent reaction by the enzyme encoded by the ychB gene. The ychB gene product phosphorylates 4-diphosphocytidyl-2C-methyl-D-erythritol, to result in formation of 4-diphosphocytidyl-2C-methyl-D-erythritol 2-phosphate. Cole et al. (supra) has reported a YchB protein in Mycobacterium tuberculosis. Recently, the ychB gene was renamed as ispE as a part of an isp gene cluster (SwissProt #P24209).

The product of the ygbB gene converts 4-diphosphocytidyl-2C-methyl-D-erythritol 2-phosphate to 2C-methyl-D-erythritol 2,4-cyclodiphosphate in Mycobacterium tuberculosis (Cole et al., supra). 2C-methyl-D-erythritol 2,4-cyclodiphosphate can be further converted into carotenoids through the carotenoid biosynthesis pathway. Recently, the ygbB gene was renamed as ispF as a part of an isp gene cluster (SwissProt #P36663). The reaction catalyzed by the YgbP enzyme is carried out in a CTP-dependent manner.

Followed by several reactions not yet characterized, isopentenyl monophosphate is formed. Isopentenyl monophosphate is converted to isopentenyl diphosphate (IPP).

It is contemplated therefore that where a specific host cell does not have the genetic machinery to produce IPP, it is well within the grasp of the skilled person to obtain any members of the above described pathway and engineer these genes into the host to produce IPP as the starting material for carotenoid biosynthesis.

Furthermore, it is expected that additional carotenoid genes from various sources could be engineered into the host microbe of choice which would further transform the carotenoid compounds produced by introduction of chimeric genes encoding one or more of the instant sequences. For example, a crtW encoding β-carotene ketolase, a crtO gene encoding β-carotene C-4 oxygenase, a crtU encoding a β-carotene desaturase, a crtA encoding a spheroidene monooxygenase, a crtC encoding a carotene hydratase, a crtD encoding a carotenoid 3,4-desaturase, or a crtF encoding a 1-OH-carotenoid methylase could be incorporated into a host microbe of choice, in addition to the instant crtEXYIB and crtZ genes, to ultimately produce canthaxanthin, astaxanthin or a variety of other carotenoid compounds.

Particularly useful downstream enzymes are the carotenoid ketolases. Carotenoid ketolases are a class of enzymes that introduce keto groups to ionone rings of the cyclic carotenoids, such as β-carotene, to produce ketocarotenoids. Ketocarotenoids include astaxanthin, canthaxanthin, adonixanthin, adonirubin, echinenone, 3-hydroxyechinenone, 3′-hydroxyechinenone, 4-keto-gamma-carotene, 4-keto-rubixanthin, 4-keto-torulene, 3-hydroxy-4-keto-torulene, deoxyflexixanthin, and myxobactone. Astaxanthin has been reported to boost immune functions in humans and reduce carcinogenesis in animals.

Vectors or cassettes useful for the transformation of suitable host cells are well known in the art. Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host.

Initiation control regions or promoters, which are useful to drive expression of the instant ORF's in the desired host cell are numerous and familiar to those skilled in the art. Virtually any promoter capable of driving these genes is suitable for the present invention including but not limited to CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, ara, tet, trp, IP_{L}, IP_{R}, T7, tac, and trc (useful for expression in Escherichia coli as well as the amy, apr, npr promoters and various phage promoters useful for expression in Bacillus.

Termination control regions may also be derived from various genes native to the preferred hosts. Optionally, a termination site may be unnecessary, however, it is most preferred if included.

Pathway Modulation

Knowledge of the sequence of the present genes will be useful in manipulating the carotenoid biosynthetic pathways in any organism having such a pathway. Methods of manipulating genetic pathways are common and well known in the art. Selected genes in a particularly pathway may be up-regulated or down-regulated by a variety of methods. Additionally, competing pathways in an organism may be eliminated or sublimated by gene disruption and similar techniques.

Once a key genetic pathway has been identified and sequenced, specific genes may be up-regulated to increase the output of the pathway. For example, additional copies of the targeted genes may be introduced into the host cell on multicopy plasmids such as pBR322. Alternatively the target genes may be modified so as to be under the control of non-native promoters. Where it is desired that a pathway operate at a particular point in a cell cycle or during a fermentation run, regulated or inducible promoters may used to replace the native promoter of the target gene. Similarly, in some cases the native or endogenous promoter may be modified to increase gene expression. For example, endogenous promoters can be altered in vivo by mutation, deletion, and/or substitution (see, Kmiec, U.S. Pat. No. 5,565,350; Zarling et al., PCT/US93/03868).

Alternatively it may be necessary to reduce or eliminate the expression of certain genes in the target pathway or in competing pathways that may serve as competing sinks for energy or carbon. Methods of down-regulating genes for this purpose have been explored. Where sequence of the gene to be disrupted is known, one of the most effective methods of gene down regulation is targeted gene disruption, where foreign DNA is inserted into a structural gene so as to disrupt transcription. This can be effected by the creation of genetic cassettes comprising the DNA to be inserted (often a genetic marker) flanked by sequence having a high degree of homology to a portion of the gene to be disrupted. Introduction of the cassette into the host cell results in insertion of the foreign DNA into the structural gene via the native DNA replication mechanisms of the cell. (See for example, Hamilton et al. J. Bacteriol. 171:4617-4622 (1989); Balbas et al. Gene 136:211-213 (1993); Gueldener et al. Nucleic Acids Res. 24:2519-2524 (1996); and Smith et al. Methods Mol. Cell. Biol. 5:270-277(1996)).

Antisense technology is another method of down regulating genes where the sequence of the target gene is known. To accomplish this, a nucleic acid segment from the desired gene is cloned and operably linked to a promoter such that the anti-sense strand of RNA will be transcribed. This construct is then introduced into the host cell and the antisense strand of RNA is produced. Antisense RNA inhibits gene expression by preventing the accumulation of mRNA that encodes the protein of interest. The person skilled in the art will know that special considerations are associated with the use of antisense technologies in order to reduce expression of particular genes. For example, the proper level of expression of antisense genes may require the use of different chimeric genes utilizing different regulatory elements known to the skilled artisan.

Although targeted gene disruption and antisense technology offer effective means of down regulating genes where the sequence is known, other less specific methodologies have been developed that are not sequence based. For example, cells may be exposed to a UV radiation and then screened for the desired phenotype. Mutagenesis with chemical agents is also effective for generating mutants and commonly used substances include chemicals that affect nonreplicating DNA (e.g., HNO₂ and NH₂OH), as well as agents that affect replicating DNA such as acridine dyes, notable for causing frameshift mutations. Specific methods for creating mutants using radiation or chemical agents are well documented in the art. See for example, Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36: 227 (1992).

Another non-specific method of gene disruption is the use of transposable elements or transposons. Transposons are genetic elements that insert randomly in DNA but can be later retrieved on the basis of sequence to determine where the insertion has occurred. Both in vivo and in vitro transposition methods are known. Both methods involve the use of a transposable element in combination with a transposase enzyme. When the transposable element or transposon is contacted with a nucleic acid fragment in the presence of the transposase, the transposable element will randomly insert into the nucleic acid fragment. The technique is useful for random mutageneis and for gene isolation, since the disrupted gene may be identified on the basis of the sequence of the transposable element. Kits for in vitro transposition are commercially available (e.g., The Primer Island Transposition Kit, available from Perkin Elmer Applied Biosystems, Branchburg, N.J., based upon the yeast Ty1 element; The Genome Priming System, available from New England Biolabs, Beverly, Mass., based upon the bacterial transposon Tn7; and the EZ::TN Transposon Insertion Systems, available from Epicentre Technologies, Madison, Wis., based upon the Tn5 bacterial transposable element).

Within the context of the present invention, it may be useful to modulate the expression of the identified carotenoid pathway by any one of the above described methods. For example, the present invention provides a number of genes encoding key enzymes in the carotenoid pathway leading to the production of pigments and smaller isoprenoid compounds. The isolated genes include the crtE, X, Y, I, B and Z genes. Where, for example it is desired to accumulate β-carotene or zeaxanthin, any of the above methods may be employed to over express lycopene cyclase (crtY) or β-carotene hydroxylase (crtZ) or any of the other upstream genes, including phytoenes desaturase, phtyoene synthase, or GGPP synthase. Similarly, in systems having functional crt genes accumulation of β-carotene or zeaxanthin may be effected by the disruption of down stream genes such as β-carotene hydroxylase (crtZ) or zeaxanthin glucosyl transferase (crtX) by any one of the methods described above.

Industrial Production

Where commercial production of the instant proteins are desired, a variety of culture methodologies may be applied. For example, large-scale production of a specific gene product overexpressed from a recombinant microbial host may be produced by both batch or continuous culture methodologies.

A classical batch culturing method is a closed system where the composition of the media is set at the beginning of the culture and not subject to artificial alterations during the culturing process. Thus, at the beginning of the culturing process the media is inoculated with the desired organism or organisms and growth or metabolic activity is permitted to occur while adding nothing to the system. Typically, however, a “batch” culture is batch with respect to the addition of carbon source and attempts are often made at controlling factors such as pH and oxygen concentration. In batch systems the metabolite and biomass compositions of the system change constantly up to the time the culture is terminated. Within batch cultures cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems. Stationary or post-exponential phase production can be obtained in other systems.

A variation on the standard batch system is the Fed-Batch system. Fed-Batch culture processes are also suitable in the present invention and comprise a typical batch system with the exception that the substrate is added in increments as the culture progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch culturing methods are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36: 227 (1992), herein incorporated by reference.

Commercial production of the instant proteins may also be accomplished with a continuous culture. Continuous cultures are an open system where a defined culture media is added continuously to a bioreactor and an equal amount of conditioned media is removed simultaneously for processing. Continuous cultures generally maintain the cells at a constant high liquid phase density where cells are primarily in log phase growth. Alternatively continuous culture may be practiced with immobilized cells where carbon and nutrients are continuously added, and valuable products, by-products or waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the culture. Methods of modulating nutrients and growth factors for continuous culture processes, as well as techniques for maximizing the rate of product formation, are well known in the art of industrial microbiology and a variety of methods are detailed by Brock, supra.

Fermentation media in the present invention must contain suitable carbon substrates. Suitable substrates may include but are not limited to monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. Additionally the carbon substrate may also be one-carbon substrates such as carbon dioxide, methane or methanol for which metabolic conversion into key biochemical intermediates has been demonstrated. In addition to one and two carbon substrates, methylotrophic organisms are also known to utilize a number of other carbon containing compounds such as methylamine, glucosamine and a variety of amino acids for metabolic activity. For example, methylotrophic yeast are known to utilize the carbon from methylamine to form trehalose or glycerol (Bellion et al., Microb. Growth C1 Compd., [Int. Symp.], 7th (1993), 415-32. Editor(s): Murrell, J. Collin; Kelly, Don P. Publisher: Intercept, Andover, UK). Similarly, various species of Candida will metabolize alanine or oleic acid (Sulter et al., Arch. Microbiol. 153:485-489 (1990)). Hence it is contemplated that the source of carbon utilized in the present invention may encompass a wide variety of carbon containing substrates and will only be limited by the choice of organism.

Recombinant Production—Plants

Plants and algae are also known to produce carotenoid compounds. The crtEXYIB and crtZ nucleic acid fragments of the instant invention may be used to create transgenic plants having the ability to express the microbial protein(s). Preferred plant hosts will be any variety that will support a high production level of the instant proteins. Suitable green plants will include, but are not limited to: soybean, rapeseed (Brassica napus, B. campestris), sunflower (Helianthus annus), cotton (Gossypium hirsutum), corn, tobacco (Nicotiana tabacum), alfalfa (Medicago sativa), wheat (Triticum sp.), barley (Hordeum vulgare), oats (Avena sativa, L), sorghum (Sorghum bicolor), rice (Oryza sativa), Arabidopsis, cruciferous vegetables (broccoli, cauliflower, cabbage, parsnips, etc.), melons, carrots, celery, parsley, tomatoes, potatoes, strawberries, peanuts, grapes, grass seed crops, sugar beets, sugar cane, beans, peas, rye, flax, hardwood trees, softwood trees, and forage grasses. Algal species include, but not limited to, commercially significant hosts such as Spirulina, Haemotacoccus, and Dunalliela. Overexpression of the carotenoid compounds may be accomplished by first constructing chimeric genes of the present invention in which the coding region(s) are operably linked to promoters capable of directing expression of a gene(s) in the desired tissues at the desired stage of development. For reasons of convenience, the chimeric genes may comprise promoter sequences and translation leader sequences derived from the same genes. 3′ Non-coding sequences encoding transcription termination signals must also be provided. The instant chimeric genes may also comprise one or more introns in order to facilitate gene expression.

Any combination of any promoter and any terminator capable of inducing expression of a coding region may be used in the chimeric genetic sequence. Some suitable examples of promoters and terminators include those from nopaline synthase (nos), octopine synthase (ocs) and cauliflower mosaic virus (CaMV) genes. One type of efficient plant promoter that may be used is a high level plant promoter. Such promoters, in operable linkage with the genetic sequences of the present invention, should be capable of promoting expression of the present gene product. High level plant promoters that may be used in this invention include, for example, 1.) the promoter of the small subunit (ss) of the ribulose-1,5-bisphosphate carboxylase from soybean (Berry-Lowe et al., J. Molecular and App. Gen., 1:483-498 (1982)); and 2.) the promoter of the chlorophyll a/b binding protein. These two promoters are known to be light-induced in plant cells (see, for example, Genetic Engineering of Plants, an Agricultural Perspective, A. Cashmore, Plenum, N.Y. (1983), pages 29-38; Coruzzi, G. et al., The Journal of Biological Chemistry, 258:1399 (1983); and Dunsmuir, P. et al., Journal of Molecular and Applied Genetics, 2:285 (1983)).

Plasmid vectors comprising the instant chimeric genes can then be constructed. The choice of plasmid vector depends upon the method that will be used to transform host plants. The skilled artisan is well aware of the genetic elements that must be present on the plasmid vector in order to successfully transform, select and propagate host cells containing the chimeric gene(s). The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus multiple events must be screened in order to obtain lines displaying the desired expression level and pattern. Such screening may be accomplished by Southern analysis of DNA blots (Southern, J. Mol. Biol. 98: 503 (1975)). Northern analysis of mRNA expression (Kroczek, J. Chromatogr. Biomed. Appl., 618 (1-2):133-145 (1993)), Western analysis of protein expression, or phenotypic analysis.

For some applications it will be useful to direct the instant proteins to different cellular compartments. It is thus envisioned that the chimeric genes described above may be further supplemented by altering the coding sequences to encode enzymes with appropriate intracellular targeting sequences such as transit sequences (Keegstra, K., Cell 56:247-253 (1989)), signal sequences or sequences encoding endoplasmic reticulum localization (Chrispeels, J. J., Ann. Rev. Plant Phys. Plant Mol. Biol. 42:21-53 (1991)), or nuclear localization signals (Raikhel, N. Plant Phys. 100:1627-1632 (1992)) added and/or with targeting sequences that are already present removed. While the references cited give examples of each of these, the list is not exhaustive and more targeting signals of utility may be discovered in the future that are useful in the invention.

It is contemplated that the present nucleotides may be used to produce gene products having enhanced or altered activity. Various methods are known for mutating a native gene sequence to produce a gene product with altered or enhanced activity including, but not limited to: error prone PCR (Melnikov et al., Nucleic Acids Research, (Feb. 15, 1999) 27(4): 1056-1062); site directed mutagenesis (Coombs et al., Proteins (1998), 259-311, 1 plate. Editor(s): Angeletti, Ruth Hogue. Publisher: Academic, San Diego, Calif.); and “gene shuffling” (U.S. Pat. No. 5,605,793; U.S. Pat. No. 5,811,238; U.S. Pat. No. 5,830,721; and U.S. Pat. No. 5,837,458, incorporated herein by reference).

The method of gene shuffling is particularly attractive due to its facile implementation, high rate of mutagenesis and ease of screening. The process of gene shuffling involves the restriction endonuclease cleavage of a gene of interest into fragments of specific size in the presence of additional populations of DNA regions of both similarity to or difference to the gene of interest. This pool of fragments will then be denatured and reannealed to create a mutated gene. The mutated gene is then screened for altered activity.

The instant microbial sequences of the present invention may be mutated and screened for altered or enhanced activity by this method. The sequences should be double stranded and can be of various lengths ranging form 50 bp to 10 kb. The sequences may be randomly digested into fragments ranging from about 10 bp to 1000 bp, using restriction endonucleases well known in the art (Maniatis, supra). In addition to the instant microbial sequences, populations of fragments that are hybridizable to all or portions of the microbial sequence may be added. Similarly, a population of fragments which are not hybridizable to the instant sequence may also be added. Typically these additional fragment populations are added in about a 10 to 20 fold excess by weight as compared to the total nucleic acid. Generally, if this process is followed, the number of different specific nucleic acid fragments in the mixture will be about 100 to about 1000. The mixed population of random nucleic acid fragments are denatured to form single-stranded nucleic acid fragments and then reannealed. Only those single-stranded nucleic acid fragments having regions of homology with other single-stranded nucleic acid fragments will reanneal. The random nucleic acid fragments may be denatured by heating. One skilled in the art could determine the conditions necessary to completely denature the double stranded nucleic acid fragments. Preferably the temperature is from 80° C. to 100° C. The nucleic acid fragments may be reannealed by cooling. Preferably the temperature is from 20° C. to 75° C. Renaturation can be accelerated by the addition of polyethylene glycol (“PEG”) or salt. A suitable salt concentration may range from 0 mM to 200 mM. The annealed nucleic acid fragments are then incubated in the presence of a nucleic acid polymerase and dNTP's (i.e., dATP, dCTP, dGTP and dTTP). The nucleic acid polymerase may be the Klenow fragment, Taq polymerase or any other DNA polymerase known in the art. The polymerase may be added to the random nucleic acid fragments prior to annealing, simultaneously with annealing or after annealing. The cycle of denaturation, renaturation and incubation in the presence of polymerase is repeated for a desired number of times. Preferably the cycle is repeated from 2 to 50 times, more preferably the sequence is repeated from 10 to 40 times. The resulting nucleic acid is a larger double-stranded polynucleotide ranging from about 50 bp to about 100 kb and may be screened for expression and altered activity by standard cloning and expression protocols (Manatis, supra).

Furthermore, a hybrid protein can be assembled by fusion of functional domains using the gene shuffling (exon shuffling) method (Nixon et al., PNAS, 94:1069-1073 (1997)). The functional domain of the instant gene can be combined with the functional domain of other genes to create novel enzymes with desired catalytic function. A hybrid enzyme may be constructed using a PCR overlap extension method and cloned into various expression vectors using the techniques well known to those skilled in art.

Description of the Preferred Embodiments

Chromosomal DNA was purified from Pantoea stewartii (ATCC No. 8199). PCR primers were designed (using the sequence from P. ananatis) to amplify a fragment containing the P. stewartii crt genes. A single fragment, approximately 6.5 kb, was amplified in the PCR reaction. This product underwent a reaction to add additional 3′ adenoside nucleotides to the fragment for TOPO cloning into pCR4-TOPO (Invitrogen, Carlsbad, Calif.). The plasmid was then transformed into E. coli and transformants were grown and screened visually for carotenoid production. Several colonies appeared to be bright yellow in color (as compared to white), indicating that they were producing a carotenoid compound.

The plasmid contained in several of these yellow colonies was reisolated, and then transposed with pGPS1.1 using the GPS-1 Genome Priming System kit (New England Biolabs, Inc., Beverly, Mass.). A number of these transposed plasmids were sequenced from each end of the transposon.

Genes encoding crtE, X, Y, I, B, and Z were identified by conducting BLAST (Basic Local Alignment Search Tool; Altschul, S. F., et al., J. Mol. Biol. 215:403-410 (1993); see also www.ncbi.nlm.nih.gov/BLAST/) searches for similarity to sequences contained in publicly available DNA sequences.

The activity of the present genes and gene products was confirmed by studies showing the effect of transposon knockouts in E. coli MG1655. By examining clones with crtE, X, Y, I, B and Z genes each individually rendered non-functional via the knock-out, it was possible to confirm the function of each gene. The wild type stain was used as a negative control. Each clone containing a specific gene knock-out was examined visually for colony pigment and tested for carotenoid concentration. Carotenoids were extracted from the cell pellets and examined by HPLC analysis.

Based on comparison of carotenoid retention times and absorption spectra, it was determined that crtZ is not expressed in the crtEXYIB construct. CrtX is proposed to encode a zeaxanthin glucosyl transferase, since knockouts of that gene accumulated β-carotene. In a similar manner, crtY mutants accumulated lycopene thereby confirming the gene's function as a lycopene cyclase. Detection of phytoene in the crtI mutant confirmed the function of the crtI gene as one encoding a phytoene dehydrogenase. Finally, loss of pigmented carotenoids in the crtE and crtB mutants indicated that these genes are essential for carotenoid synthesis. This observation is consistent with the proposed function of crtB encoding a prephytoene pyrophosphate synthase and crtE encoding a geranylgeranyl pyrophosphate synthetase.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

General Methods

Standard recombinant DNA and molecular cloning techniques used in the Examples are well known in the art and are described by: Sambrook, J., Fritsch, E. F. and Maniatis, T. in Molecular Cloning: A Laboratory Manual; Cold Spring Harbor Laboratory Press: Cold Spring Harbor, (1989) (Maniatis); by T. J. Silhavy, M. L. Bennan, and L. W. Enquist, in Experiments with Gene Fusions, Cold Spring Harbor Laboratory, Cold Spring Harbor, N.Y. (1984); and by Ausubel, F. M. et al., in Current Protocols in Molecular Biology, published by Greene Publishing Assoc. and Wiley-Interscience (1987).

Materials and methods suitable for the maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)) or by Thomas D. Brock in Biotechnology: A Textbook of Industrial Microbiology, Second Edition, Sinauer Associates, Inc., Sunderland, Mass. (1989). All reagents, restriction enzymes and materials used for the growth and maintenance of bacterial cells were obtained from Aldrich Chemicals (Milwaukee, Wis.), DIFCO Laboratories (Detroit, Mich.), GIBCO/BRL (Gaithersburg, Md.), or Sigma Chemical Company (St. Louis, Mo.) unless otherwise specified.

Manipulations of genetic sequences were accomplished using the suite of programs available from the Genetics Computer Group, Inc. (Wisconsin Package Version 9.0, Genetics Computer Group (GCG), Madison, Wis.). Where the GCG program “Pileup” was used the gap creation default value of 12 and the gap extension default value of 4 were used. Where the GCG “Gap” or “Bestfit” programs were used the default gap creation penalty of 50 and the default gap extension penalty of 3 were used. In any case where GCG program parameters were not prompted for, in these or any other GCG program, default values were used.

The meaning of abbreviations is as follows: “h” means hour(s), “min” means minute(s), “sec” means second(s), “d” means day(s), “mL” means milliliters, “L” means liters, “kB” means kilobase(s), and “nm” means nanometers.

### Example 1

### Cloning of Genes from Pantoea stewartii

Primers were designed using the sequence from Erwinia uredovora to amplify a fragment by PCR containing the crt genes. These sequences included 5′-3′:

| | |
|---|---|
| ATGACGGTCTGCGCAAAAAAACACG | SEQ ID NO: 13 |
| GAGAAATTATGTTGTGGATTTGGAATGC | SEQ ID NO: 14 |

Chromosomal DNA was purified from Pantoea stewartii (ATCC No. 8199) and Pfu Turbo polymerase (Stratagene, La Jolla, Calif.) was used in a PCR amplification reaction under the following conditions: 94° C., 5 min; 94° C. (1 min)-60° C. (1 min)-72° C. (10 min) for 25 cycles, and 72° C. for 10 min. A single product of approximately 6.5 kb was observed following gel electrophoresis. Taq polymerase (Perkin Elmer) was used in a 10 min 72° C. reaction to add additional 3′ adenosine nucleotides to the fragment for TOPO cloning into pCR4-TOPO (Invitrogen, Carlsbad, Calif.). Following transformation to E. coli DH5α (Life Technologies, Rockville, Md.) by electroporation, several colonies appeared to be bright yellow in color, indicating that they were producing a carotenoid compound. Following plasmid isolation as instructed by the manufacturer using the Qiagen miniprep kit (Valencia, Calif.), the plasmid containing the 6.5 kb amplified fragment was transposed with pGPS1.1 using the GPS-1 Genome Priming System kit (New England Biolabs, Inc., Beverly, Mass.). A number of these transposed plasmids were sequenced from each end of the transposon. Sequence was generated on an ABI Automatic sequencer using dye terminator technology (U.S. Pat. No. 5,366,860; EP 272007) using transposon specific primers. Sequence assembly was performed with the Sequencher program (Gene Codes Corp., Ann Arbor, Mich.).

### Example 2

### Identification and Characterization of Bacterial Genes

Genes encoding crtE, X, Y, I, B, and Z were identified by conducting BLAST (Basic Local Alignment Search Tool; Altschul, S. F., et al., J. Mol. Biol. 215:403-410 (1993); see also www.ncbi.nlm.nih.gov/BLAST/) searches for similarity to sequences contained in the BLAST “nr” database (comprising all non-redundant GenBank CDS translations, sequences derived from the 3-dimensional structure Brookhaven Protein Data Bank, the SWISS-PROT protein sequence database, EMBL, and DDBJ databases). The sequences obtained in Example 1 were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish, W. and States, D. J. (1993) Nature Genetics 3:266-272) provided by the NCBI.

All comparisons were done using either the BLASTNnr or BLASTXnr algorithm. The results of the BLAST comparison are given in Table 2, which summarizes the sequences to which each gene has the most similarity. Table 2 displays data based on the BLASTXnr algorithm with values reported in expect values. The Expect value estimates the statistical significance of the match, specifying the number of matches with a given score that are expected in a search of a database of this size absolutely by chance.

TABLE 2

| ORF | Gene | | SEQ ID | SEQ ID | % | % | | |
|---|---|---|---|---|---|---|---|---|
| Name | Name | Similarity Identified | base | Peptide | Identity^{a} | Similarity^{b} | E-value^{c} | Citation |
| 1 | crtE | Geranylgeranyl pryophosphate synthetase (or | 1 | 2 | 83 | 88 | e−137 | Misawa et |
| | | GGPP synthetase, or farnesyltranstransferase) | | | | | | al., J. |
| | | EC 2.5.1.29 | | | | | | Bacteriol. |
| | | gi\|117509\|sp\|P21684\|CRTE_PANAN | | | | | | 172 (12), |
| | | GERANYLGERANYL PYROPHOSPHATE | | | | | | 6704-6712 |
| | | SYNTHETASE (GGPP SYNTHETASE) | | | | | | (1990) |
| | | (FARNESYLTRANSTRANSFERASE) | | | | | | |
| 2 | crtX | Zeaxanthin glucosyl transferase EC 2.4.1.- | 3 | 4 | 75 | 79 | 0.0 | Lin et al., |
| | | gi\|1073294\|pir∥S52583 crtX protein - Erwinia | | | | | | Mol. Gen. |
| | | herbicola | | | | | | Genet. 245 |
| | | | | | | | | (4), 417-423 |
| | | | | | | | | (1994) |
| 3 | crtY | Lycopene cyclase | 5 | 6 | 83 | 91 | 0.0 | Lin et al., |
| | | gi\|1073295\|pir∥S52585 lycopene cyclase - | | | | | | Mol. Gen. |
| | | Erwinia herbicola | | | | | | Genet. 245 |
| | | | | | | | | (4), 417-423 |
| | | | | | | | | (1994) |
| 4 | crtl | Phytoene desaturaseEC 1.3.-.- | 7 | 8 | 89 | 91 | 0.0 | Lin et al., |
| | | gi\|1073299\|pir∥S52586 phytoene | | | | | | Mol. Gen. |
| | | dehydrogenase (EC 1.3.-.-) - Erwinia herbicola | | | | | | Genet. 245 |
| | | | | | | | | (4), 417-423 |
| | | | | | | | | (1994) |
| 5 | crtB | Phytoene synthaseEC2.5.1.- | 9 | 10 | 88 | 92 | e−150 | Lin et al., |
| | | gi\|1073300\|pir∥S52587 prephytoene | | | | | | Mol. Gen. |
| | | pyrophosphate synthase - Erwinia herbicola | | | | | | Genet. 245 |
| | | | | | | | | (4), 417-423 |
| | | | | | | | | (1994) |
| 6 | crtZ | β-carotene hydroxylase | 11 | 12 | 88 | 91 | 3e−88 | Misawa et |
| | | gi\|117526\|sp\|P21688\|CRTZ_PANAN BETA- | | | | | | al., J. |
| | | CAROTENE HYDROXYLASE | | | | | | Bacteriol. |
| | | | | | | | | 172 (12), |
| | | | | | | | | 6704-6712 |
| | | | | | | | | (1990) |

^{a}% Identity is defined as percentage of amino acids that are identical between the two proteins.
^{b}% Similarity is defined as percentage of amino acids that are identical or conserved between the two proteins.
^{c}Expect value. The Expect value estimates the statistical significance of the match, specifying the number of matches, with a given score, that are expected in a search of a database of this size absolutely by chance.

### Example 3

### Analysis of Gene Function by Transposon Mutagenesis

Several plasmids carrying transposons which were inserted into each coding region including crtE, crtX, crtY, crtI, crtB, and crtZ were chosen using sequence data generated in Example 1. These plasmid variants were transformed to E. coli MG1655 and grown in 100 mL Luria-Bertani broth in the presence of 100 ug/mL ampicillin. Cultures were grown for 18 h at 26° C., and the cells were harvested by centrifugation. Carotenoids were extracted from the cell pellets using 10 mL of acetone. The acetone was dried under nitrogen and the carotenoids were resuspended in 1 mL of methanol for HPLC analysis. A Beckman System Gold® HPLC with Beckman Gold Nouveau Software (Columbia, Md.) was used for the study. The crude extraction (0.1 mL) was loaded onto a 125×4 mm RP8 (5 μm particles) column with corresponding guard column (Hewlett-Packard, San Fernando, Calif.). The flow rate was 1 mL/min, while the solvent program used was: 0-11.5 min 40% water/60% methanol; 11.5-20 min 100% methanol; and 20-30 min 40% water/60% methanol. The spectrum data were collected by a Beckman photodiode array detector (model 168).

In the wild-type clone with wild-type crtEXYIBZ, the carotenoid was found to have a retention time of 15.8 min and an absorption spectra of 450 nm, 475 nm. This was the same as the β-carotene standard. This suggested that the crtZ gene organized in the opposite orientation was not expressed in this construct. The transposon insertion in crtZ had no effect as expected (data not shown).

HPLC spectral analysis also revealed that a clone with transposon insertion in crtX also produced β-carotene. This is consistent with the proposed function of crtX encoding a zeaxanthin glucosyl transferase enzyme at a later step of the carotenoid pathway following synthesis of β-carotene.

The transposon insertion in crtY did not produce β-carotene. The carotenoid's elution time (15.2 min) and absorption spectra (443 nm, 469 nm, 500 nm) agree with those of the lycopene standard. Accumulation of lycopene in the crtY mutant confirmed the role of crtY as a lycopene cyclase encoding gene.

The crtI extraction, when monitored at 286 nm, had a peak with retention time of 16.3 min and with absorption spectra of 276 nm, 286 nm, and 297 nm, which agrees with the reported spectrum for phytoene. Detection of phytoene in the crtI mutant confirmed the function of the crtI gene as one encoding a phytoene dehydrogenase enzyme.

The extraction of the crtE mutant, crtB mutant and crtI mutant was clear. Loss of pigmented carotenoids in these mutants indicated that both the crtE gene and crtB gene are essential for carotenoid synthesis. No carotenoid was observed in either mutant, which is consistent with the proposed function of crtB encoding a prephytoene pyrophosphate synthase and crtE encoding a geranylgeranyl pyrophosphate synthetase. Both enzymes are required for β-carotene synthesis.

Results of the transposon mutagenesis experiments are shown below in Table 3. The site of transposon insertion into the gene cluster crtEXYIB is recorded, along wih the color of the E. coli colonies observed on LB plates, the identity of the caretenoid compound (as determined by HPLC spectral analysis), and the experimentally assigned function of each gene.

TABLE 3

| Transposon | | Carotenoid observed | Assigned gene |
|---|---|---|---|
| insertion site | Colony color | by HPLC | function |
| Wild Type (with no | Yellow | β-carotene | |
| transposon | | | |
| insertion) | | | |
| crtE | White | None | Geranylgeranyl |
| | | | pyrophosphate |
| | | | synthetase |
| crtB | White | None | Prephytoene |
| | | | pyrophosphate |
| | | | synthase |
| crtI | White | Phytoene | Phytoene |
| | | | dehydrogenase |
| crtY | Pink | Lycopene | Lycopene |
| | | | cyclase |
| crtZ | Yellow | β-carotene | β-carotene |
| | | | hyroxylase |
| crtX | Yellow | β-carotene | Zeaxanthin |
| | | | glucosyl |
| | | | transferase |

What is claimed is:

1. An isolated nucleic acid molecule encoding a carotenoid biosynthetic enzyme, selected from the group consisting of:
(a) an isolated nucleic acid molecule encoding the amino acid sequence selected from the group consisting of SEQ ID NOs:2, 4, 6, 8, 10, and 12;
(b) an isolated nucleic acid molecule that hybridizes with (a) under the following hybridization conditions: 0.1×SSC, 0.1% SDS, 65° C. and washed with 2×SSC, 0.1% SDS followed by 0.1×SSC, 0.1% SDS; and
(c) an isolated nucleic acid molecule that is complementary to (a) or (b).

2. The isolated nucleic acid molecule of claim 1 selected from the group consisting of SEQ ID NOs:1, 3, 5, 7, 9, and 11.

3. A polypeptide encoded by the isolated nucleic acid molecule of claim 1.

4. The polypeptide of claim 3 selected from the group consisting of SEQ ID NOs:2, 4, 6, 8, 10, and 12.

5. An isolated nucleic acid molecule comprising a first nucleotide sequence encoding a geranylgeranyl pyrophosphate synthase enzyme of at least 303 amino acids that has at least 83% identity based on the Smith-Waterman method of alignment when compared to a polypeptide having the sequence as set forth in SEQ ID NO:2;
or a second nucleotide sequence comprising the complement of the first nucleotide sequence.

6. An isolated nucleic acid molecule comprising a first nucleotide sequence encoding a zeaxanthin glucosyl transferase enzyme of at least 431 amino acids that has at least 75% identity based on the Smith-Waterman method of alignment when compared to a polypeptide having the sequence as set forth in SEQ ID NO:4;
or a second nucleotide sequence comprising the complement of the first nucleotide sequence.

7. An isolated nucleic acid molecule comprising a first nucleotide sequence encoding a lycopene cyclase enzyme of at least 382 amino acids that has at least 83% identity based on the Smith-Waterman method of alignment when compared to a polypeptide having the sequence as set forth in SEQ ID NO:6;
or a second nucleotide sequence comprising the complement of the first nucleotide sequence.

8. An isolated nucleic acid molecule comprising a first nucleotide sequence encoding a phytoene desaturase enzyme of at least 492 amino acids that has at least 89% identity based on the Smith-Waterman method of alignment when compared to a polypeptide having the sequence as set forth in SEQ ID NO:8;
or a second nucleotide sequence comprising the complement of the first nucleotide sequence.

9. An isolated nucleic acid molecule comprising a first nucleotide sequence encoding a phytoene synthase enzyme of at least 296 amino acids that has at least 88% identity based on the Smith-Waterman method of alignment when compared to a polypeptide having the sequence as set forth in SEQ ID NO:10;
or a second nucleotide sequence comprising the complement of the first nucleotide sequence.

10. An isolated nucleic acid molecule comprising a first nucleotide sequence encoding a β-carotene hydroxylase enzyme of at least 175 amino acids that has at least 88% identity based on the Smith-Waterman method of alignment when compared to a polypeptide having the sequence as set forth in SEQ ID NO:12;
or a second nucleotide sequence comprising the complement of the first nucleotide sequence.

11. A chimeric gene comprising the isolated nucleic acid molecule of any one of claims 1 or 5-10 operably linked to suitable regulatory sequences.

12. A transformed host cell comprising the chimeric gene of claim 11.

13. The transformed host cell of claim 12 wherein the host cell is selected from the group consisting of bacteria, yeast, filamentous fungi, algae, and green plants.

14. The transformed host cell of claim 13 wherein the host cell is selected from the group consisting of Aspergillus, Trichoderma, Saccharomyces, Pichia, Candida, Hansenula, Yarrowia, Rhodosporidium, Lipomyces, Salmonella, Bacillus, Acinetobacter, Zymomonas, Agrobacterium, Flavobacterium, Rhodobacter, Rhodococcus, Streptomyces, Brevibacterium, Corynebacteria, Mycobacterium, Escherichia, Pantoea, Pseudomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium and Klebsiella.

15. The transformed host cell of claim 13 wherein the host cell is selected from the group consisting of soybean, rapeseed, sunflower, cotton, corn, tobacco, alfalfa, wheat, barley, oats, sorghum, rice, Arabidopsis, cruciferous vegetables, melons, carrots, celery, parsley, tomatoes, potatoes, strawberries, peanuts, grapes, grass seed crops, sugar beets, sugar cane, beans, peas, rye, flax, hardwood trees, softwood trees, and forage grasses.

16. A method of obtaining a nucleic acid molecule encoding a carotenoid biosynthetic enzyme comprising:
(a) probing a genomic library with the nucleic acid molecule of any one of claims 1 or 5-10;
(b) identifying a DNA clone that hybridizes with the nucleic acid molecule of any one of claims 1 or 5-10; and
(c) sequencing the genomic fragment that comprises the clone identified in step (b),
wherein the sequenced genomic fragment encodes a carotenoid biosynthetic enzyme.

17. A method of obtaining a nucleic acid molecule encoding a carotenoid biosynthetic enzyme comprising:
(a) synthesizing at least one oligonucleotide primer corresponding to a portion of the sequence selected from the group consisting of SEQ ID NOs:1, 3, 5, 7, 9, 11; and
(b) amplifying an insert present in a cloning vector using the oligonucleotide primer of step (a);
wherein the amplified insert encodes a portion of an amino acid sequence encoding a carotenoid biosynthetic enzyme.

18. The product of the method of claims 16 or 17.

19. A method for the production of carotenoid compounds comprising:
(a) providing a transformed host cell comprising:
(i) suitable levels of isopentenyl pyrophosphate; and
(ii) a set of nucleic acid molecules encoding the enzymes selected from the group consisting of SEQ ID NOs:2, 4, 6, 8, 10, and 12 under the control of suitable regulatory sequences;
(b) contacting the host cell of step (a) under suitable growth conditions with an effective amount of a fermentable carbon substrate whereby a carotenoid compound is produced.

20. A method according to claim 19 wherein the transformed host cell is selected from the group consisting of bacteria, yeast, filamentous fungi, algae, and green plants.

21. A method according to claim 20 wherein the transformed host cell is selected from the group consisting of Aspergillus, Trichoderma, Saccharomyces, Pichia, Candida, Hansenula, Yarrowia, Rhodosporidium, Lipomyces, Salmonella, Bacillus, Acinetobacter, Zymomonas, Agrobacterium, Flavobacterium, Rhodobacter, Rhodococcus, Streptomyces, Brevibacterium, Corynebacteria, Mycobacterium, Escherichia, Pantoea, Pseudomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium and Klebsiella.

22. A method according to claim 20 wherein the transformed host cell is selected from the group consisting of soybean, rapeseed, sunflower, cotton, corn, tobacco, alfalfa, wheat, barley, oats, sorghum, rice, Arabidopsis, cruciferous vegetables, melons, carrots, celery, parsley, tomatoes, potatoes, strawberries, peanuts, grapes, grass seed crops, sugar beets, sugar cane, beans, peas, rye, flax, hardwood trees, softwood trees, and forage grasses.

23. A method of regulating carotenoid biosynthesis in an organism comprising, over-expressing at least one carotenoid gene selected from the group consisting of SEQ ID NOs:1, 3, 5, 7, 9, and 11 in an organism such that the carotenoid biosynthesis is altered in the organism.

24. A method according to claim 23 wherein said carotenoid gene is over-expressed on a multicopy plasmid.

25. A method according to claim 23 wherein said carotenoid gene is operably linked to an inducible or regulated promoter.

26. A method according to claim 23 wherein said carotenoid gene is expressed in antisense orientation.

27. A method according to claim 23 wherein said carotenoid gene is disrupted by insertion of foreign DNA into the coding region.

28. A mutated gene encoding a carotenoid enzyme having an altered biological activity produced by a method comprising the steps of:
(i) digesting a mixture of nucleotide sequences with restriction endonucleases wherein said mixture comprises:
a) an isolated nucleic acid molecule encoding a carotenoid biosynthetic enzyme selected from the group consisting of SEQ ID NOs:1, 3, 5, 7, 9, and 11;
b) a first population of nucleotide fragments which will hybridize to said isolated nucleic acid molecules of step (a); and
c) a second population of nucleotide fragments which will not hybridize to said isolated nucleic acid molecules of step (a);
wherein a mixture of restriction fragments are produced;
(ii) denaturing said mixture of restriction fragments;
(iii) incubating the denatured said mixture of restriction fragments of step (ii) with a polymerase; and
(iv) repeating steps (ii) and (iii), wherein a mutated carotenoid gene is produced encoding a protein having an altered biological activity.
